(19) United States
(12) Patent Application Publication
(10) Pub. No.: US 2022/0002332 A1
(43) Pub. Date: Jan. 6, 2022

(54) SCUTELLARIN AMIDE DERIVATIVES, AND PREPARATION METHODS AND USES THEREOF

(71) Applicant: HEILONGJIANG BAYI AGRICULTURAL UNIVERSITY, Daqing City (CN)

(72) Inventors: Chenghao JIN, Daqing City (CN); Tong Han, Daqing City (CN); Dahong Li, Daqing City (CN); Huiming Hua, Daqing City (CN); Chunyu Jiang, Daqing City (CN); Xinmiao Zhang, Daqing City (CN); Xin Xu, Daqing City (CN)

(73) Assignee: HEILONGJIANG BAYI AGRICULTURAL UNIVERSITY, Daqing City (CN)

(21) Appl. No.: 17/360,502

(22) Filed: Jun. 28, 2021

(30) Foreign Application Priority Data
Jul. 3, 2020 (CN) 202010630953.1

(51) Int. Cl. C07H 17/07; C07H 1/00

(52) U.S. Cl. CPC C07H 17/07; C07H 1/00

(57) ABSTRACT

The present disclosure discloses scutellarin amide derivatives and preparation methods and uses thereof, which belongs to the field of natural drugs and medicinal chemistry. The scutellarin amide derivatives according to the present disclosure and pharmaceutically acceptable salts thereof have a structure as shown in the following general formula I:

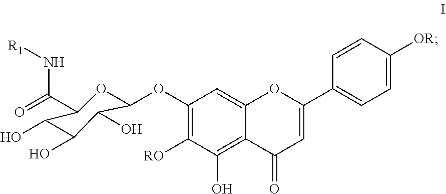

scutellarin derivatives, which are prepared by amidation at the glycosylcarboxyl site, can be used in the manufacture of anti-tumor drugs, which have a good effect against tumor cell proliferation.

### CROSS REFERENCE TO RELATED APPLICATION(S)

This patent application claims the benefit and priority of Chinese Patent Application No. 202010630953.1, filed on Jul. 3, 2020, the disclosure of which is incorporated by reference herein in its entirety as part of the present application.

### TECHNICAL FIELD

The present disclosure belongs to the field of medicinal chemistry, and specifically relates to derivatives of scutellarin modified by amidation at the glycosylcarboxyl site of scutellarin, and preparation methods and uses thereof. Specifically, it relates to these scutellarin amide derivatives that are amidated at the glycosylcarboxyl site, and preparation methods thereof and their uses in anti-tumor.

### BACKGROUND ART

So far, cancer is still one of the most dominant diseases threatening human health all around the world. In China, the mortality rate of malignant tumors has been on the rise in the past ten years, and it has become one of the major public health problems that seriously threaten the health of people in China. It is urgent to develop new anti-tumor drugs.

Natural products are an important source of drug discovery. Among the drugs on the market, many successful drugs are derived from natural products either directly or indirectly. Therefore, in nature, it is important to find and obtain anti-tumor candidate compounds with better activity, lower toxicity and more stable properties.

Scutellarin is an active ingredient of flavonoid type, which is a light yellow powder extracted and separated from the dried whole plant of Compositae Erigeron breviscapus (Vant.) Hand-Mazz. Modern pharmacological studies have shown that scutellarin has a wide range of pharmacological activities, including blood vessel expansion, blood flow increase, anticoagulation, platelet aggregation inhibition, tumor cell proliferation inhibition, nerve cell protection, and the like. In recent years, there is an increasingly deep research on the anti-tumor effect of scutellarin, and related studies have shown that scutellarin has a good inhibitory effect on a variety of tumor cell lines. Examples of the tumor cell lines include human leukemia cells, breast cancer cells, liver cancer cells, colon cancer cells, human tongue cancer cells, etc. Scutellarin, as a common flavonoid compound, has a wide range of sources and exists in many edible plants, which lays a good foundation for the development of high-efficiency and low-toxic anti-tumor drugs.

### SUMMARY

In the present disclosure, scutellarin is used as the lead compound, and different types of anilines and aliphatic amines are linked to the carboxyl position of scutellarin through multi-step chemical reactions. Scutellarin amide derivatives are designed and synthesized, and the anti-tumor bioactivity of the synthetic derivatives is tested.

The technical problem to be solved by the present disclosure is to find scutellarin amide derivatives with good anti-tumor activity, and further provide a pharmaceutical composition for treating tumors and other diseases or disorders. In the present disclosure, scutellarin is amidated to obtain derivatives, which improves its anti-tumor effect, enhances the pharmacokinetic properties of scutellarin, and increases its stability.

In order to solve the above technical problems, the present disclosure provides the following technical solutions:

The general formula I shows a scutellarin amide derivative and a pharmaceutically acceptable salt thereof:

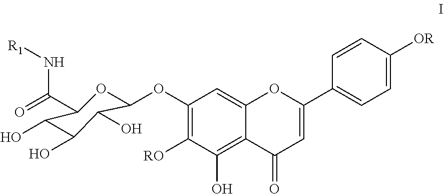

wherein R is a substituted or unsubstituted C₁-C₁₂ alkyl group, a substituted or unsubstituted benzyl group on the benzene ring, and the substituent is a C₁-C₄ alkyl group, a C₁-C₄ alkoxy group; R₁ is a substituted or unsubstituted C₁-C₁₂ alkyl group, a substituted or unsubstituted phenyl group, and the substituent is halogen, a C₁-C₄ alkyl group or a C₁-C₄ alkoxy group.

Further, R is a substituted or unsubstituted C₁-C₆ alkyl group, a substituted or unsubstituted benzyl group on the benzene ring, and the substituent is a C₁-C₄ alkyl group, a C₁-C₄ alkoxy group; R₁ is a substituted or unsubstituted C₁-C₆ alkyl group, a substituted or unsubstituted phenyl group, and the substituent is halogen, a C₁-C₄ alkyl group or a C₁-C₄ alkoxy group.

Preferably, R is methyl, ethyl, n-propyl, isopropyl, n-butyl, isobutyl or benzyl; R₁ is ethyl, n-propyl, isopropyl, n-butyl, n-pentyl, n-hexyl, cyclohexyl, phenyl, 4-chlorophenyl, 3-chlorophenyl, 2-chlorophenyl, 4-methylphenyl, 3-methylphenyl, 2-methylphenyl, 4-hydroxyphenyl, 3-hydroxyphenyl, 2-hydroxyphenyl, 4-methoxyphenyl, 3-methoxyphenyl, 2-methoxyphenyl.

More preferably, R is methyl or benzyl; R₁ is n-propyl, isopropyl, n-hexyl, cyclohexyl, phenyl, 4-chlorophenyl, 3-chlorophenyl, 4-methylphenyl, 3-methylphenyl, 2-methylphenyl, 4-hydroxyphenyl, 2-hydroxyphenyl, 4-methoxyphenyl, 3-methoxyphenyl, 2-methoxyphenyl.

Further, the present disclosure preferably includes the following derivatives and pharmaceutically acceptable salts thereof:

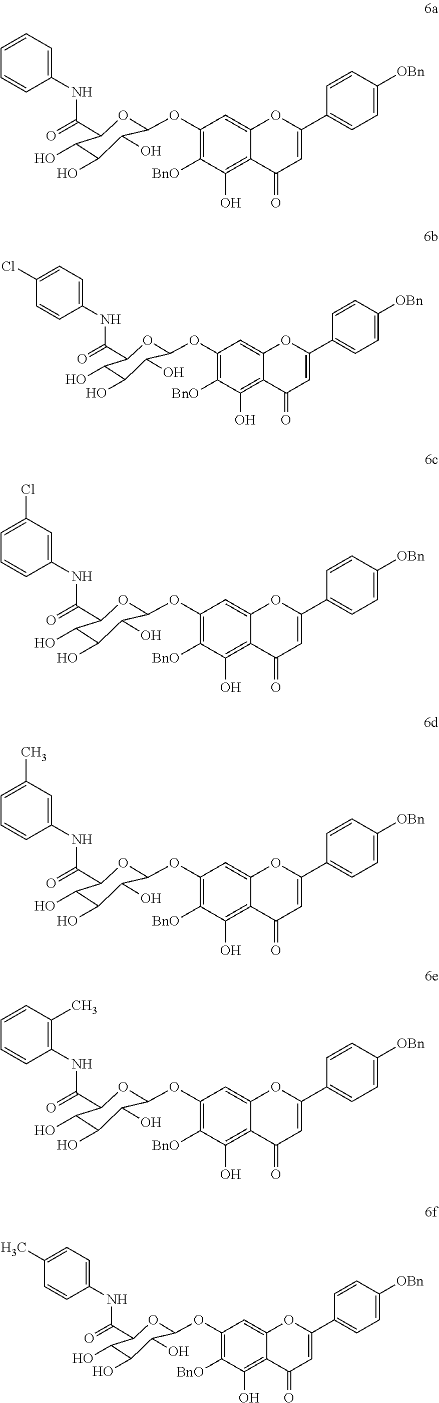

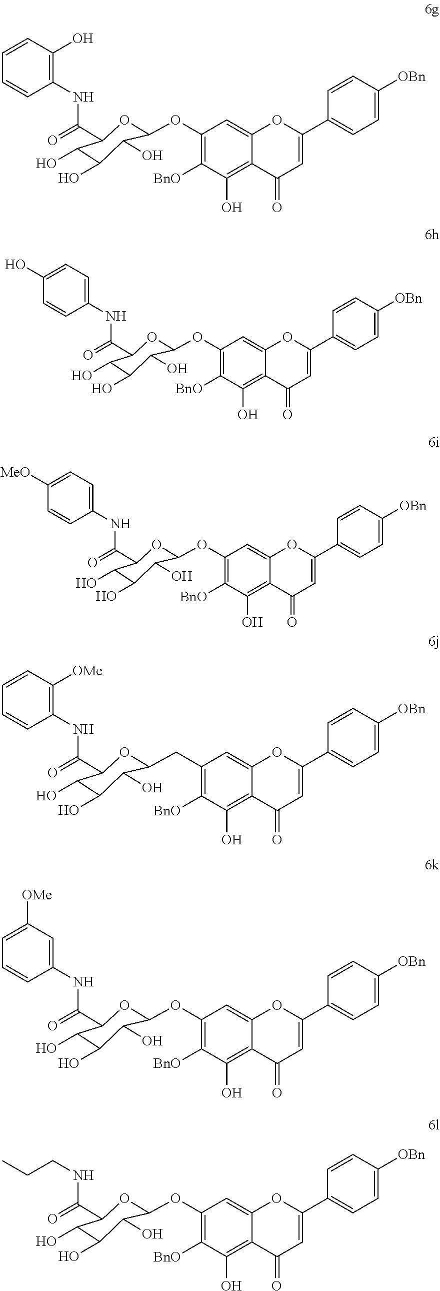

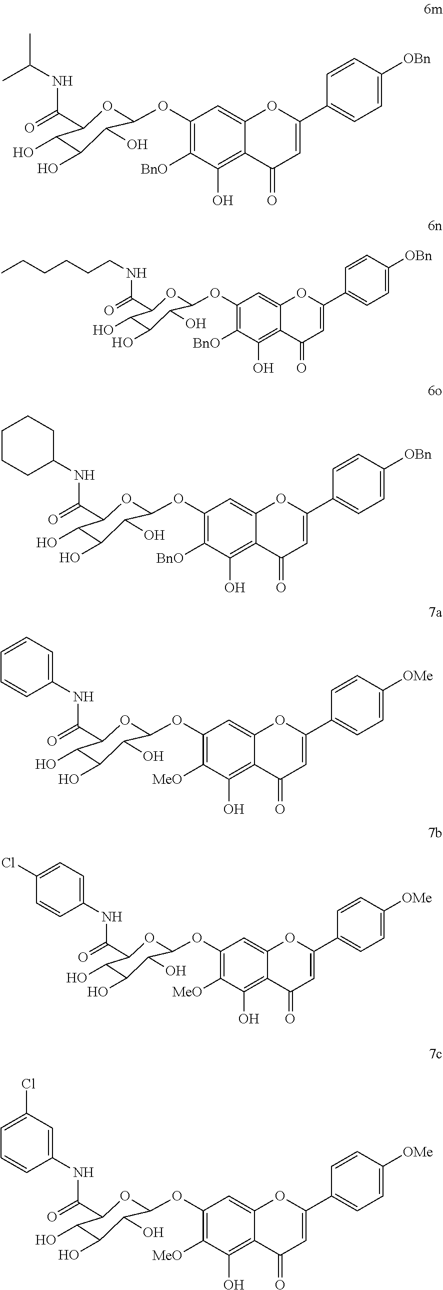 [SMILES: CC(C)NC(=O)C1OC(Oc2cc3oc(-c4ccc(OCc5ccccc5)cc4)cc(=O)c3c(O)c2OCc2ccccc2)C(O)C(O)C1O ; CCCCCCNC(=O)C1OC(Oc2cc3oc(-c4ccc(OCc5ccccc5)cc4)cc(=O)c3c(O)c2OCc2ccccc2)C(O)C(O)C1O ; O=C(NC1CCCCC1)C1OC(Oc2cc3oc(-c4ccc(OCc5ccccc5)cc4)cc(=O)c3c(O)c2OCc2ccccc2)C(O)C(O)C1O ; COc1ccc(-c2cc(=O)c3c(O)c(OC)c(OC4OC(C(=O)Nc5ccccc5)C(O)C(O)C4O)cc3o2)cc1 ; COc1ccc(-c2cc(=O)c3c(O)c(OC)c(OC4OC(C(=O)Nc5ccc(Cl)cc5)C(O)C(O)C4O)cc3o2)cc1 ; COc1ccc(-c2cc(=O)c3c(O)c(OC)c(OC4OC(C(=O)Nc5cccc(Cl)c5)C(O)C(O)C4O)cc3o2)cc1]

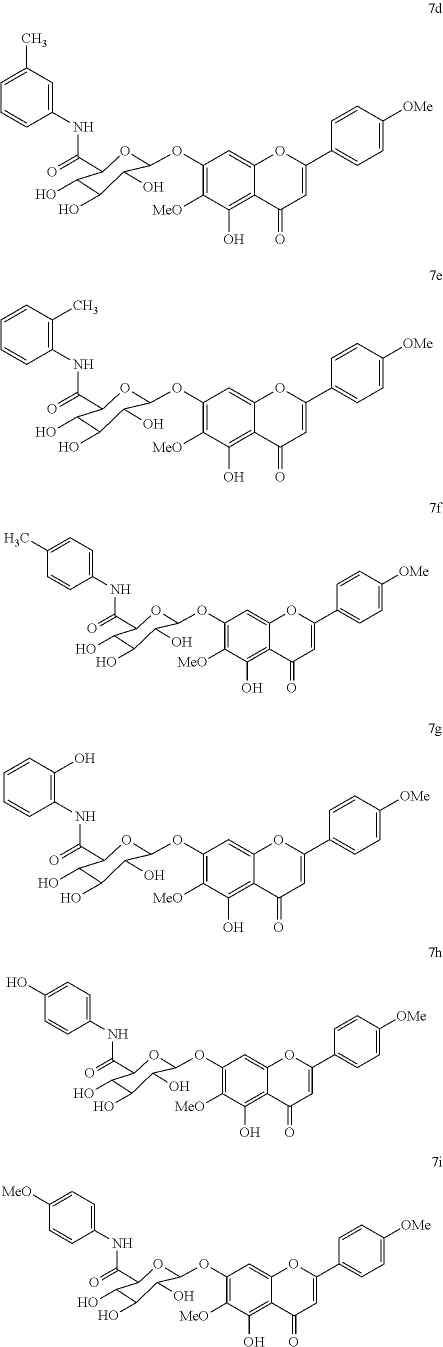 [SMILES: COc1ccc(-c2cc(=O)c3c(O)c(OC)c(OC4OC(C(=O)Nc5cccc(C)c5)C(O)C(O)C4O)cc3o2)cc1 ; COc1ccc(-c2cc(=O)c3c(O)c(OC)c(OC4OC(C(=O)Nc5ccccc5C)C(O)C(O)C4O)cc3o2)cc1 ; COc1ccc(-c2cc(=O)c3c(O)c(OC)c(OC4OC(C(=O)Nc5ccc(C)cc5)C(O)C(O)C4O)cc3o2)cc1 ; COc1ccc(-c2cc(=O)c3c(O)c(OC)c(OC4OC(C(=O)Nc5ccccc5O)C(O)C(O)C4O)cc3o2)cc1 ; COc1ccc(-c2cc(=O)c3c(O)c(OC)c(OC4OC(C(=O)Nc5ccc(O)cc5)C(O)C(O)C4O)cc3o2)cc1 ; COc1ccc(NC(=O)C2OC(Oc3cc4oc(-c5ccc(OC)cc5)cc(=O)c4c(O)c3OC)C(O)C(O)C2O)cc1]

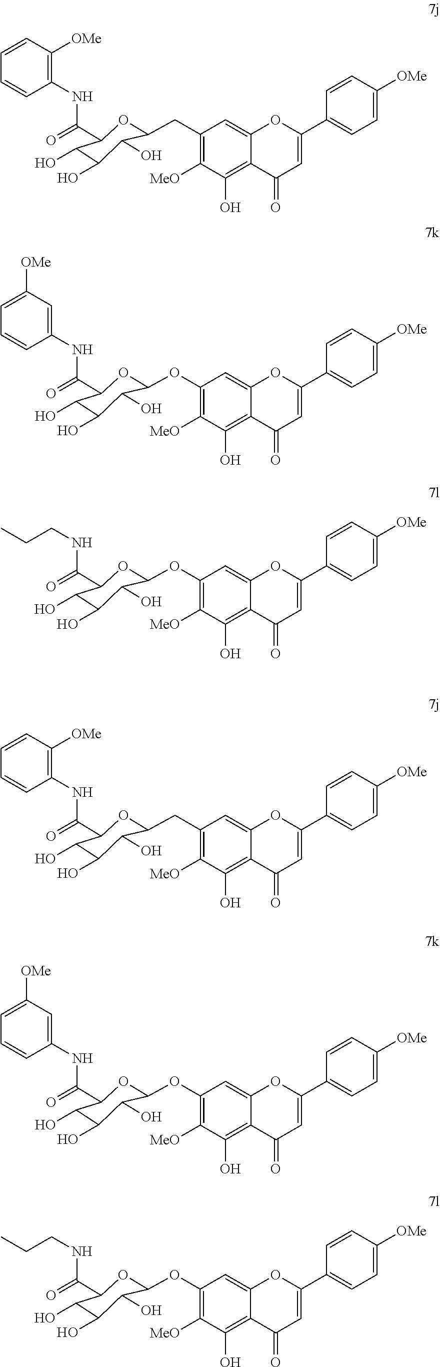 [SMILES: COc1ccc(-c2cc(=O)c3c(O)c(OC)c(CC4OC(C(=O)Nc5ccccc5OC)C(O)C(O)C4O)cc3o2)cc1 ; COc1ccc(-c2cc(=O)c3c(O)c(OC)c(OC4OC(C(=O)Nc5cccc(OC)c5)C(O)C(O)C4O)cc3o2)cc1 ; CCCNC(=O)C1OC(Oc2cc3oc(-c4ccc(OC)cc4)cc(=O)c3c(O)c2OC)C(O)C(O)C1O ; COc1ccc(-c2cc(=O)c3c(O)c(OC)c(CC4OC(C(=O)Nc5ccccc5OC)C(O)C(O)C4O)cc3o2)cc1 ; COc1ccc(-c2cc(=O)c3c(O)c(OC)c(OC4OC(C(=O)Nc5cccc(OC)c5)C(O)C(O)C4O)cc3o2)cc1 ; CCCNC(=O)C1OC(Oc2cc3oc(-c4ccc(OC)cc4)cc(=O)c3c(O)c2OC)C(O)C(O)C1O]

The derivatives of the general formula I of the present disclosure can be prepared by the following method:

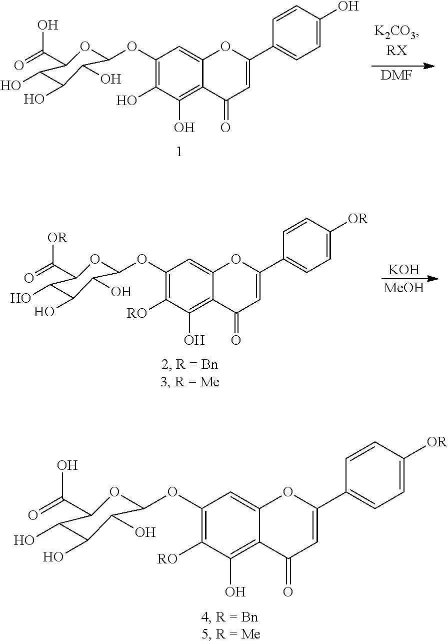

Step 1: Scutellarin is reacted with a corresponding halogenated hydrocarbon in the presence of K₂CO₃/DMF for 24-48 hours, and then subjected to a silica gel column chromatography with 10:1 to 50:1 dichloromethane-methanol as the eluent to obtain intermediates (2, 3); subsequently, the intermediates 2, 3 are hydrolyzed in the presence of KOH/MeOH to obtain target compounds (4, 5);

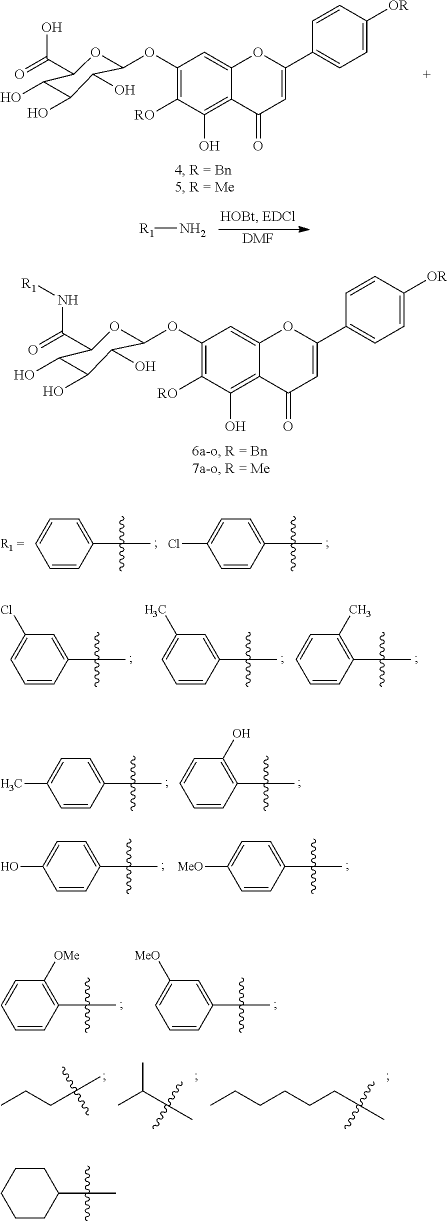

Step 2: The glycosylcarboxyl position of the target compounds 4, 5 is reacted with an aniline substituted by different substituents and substituted at different positions and an aliphatic amine with different side chains at room temperature for 12-24 hours in the presence of HOBt, EDCI catalysts, and then subjected to a silica gel column chromatography with 10:1 to 100:1 dichloromethane-methanol as the eluent to obtain target compounds 6a-o and 7a-o.

Provided is a use of a scutellarin amide derivative represented by the general formula I and a pharmaceutically acceptable salt thereof as an active ingredient in the preparation of a medicament for the treatment of tumor diseases.

Provided is a use of a scutellarin amide derivative represented by the general formula I and a pharmaceutically acceptable salt thereof as an active ingredient in the preparation of a medicament for the treatment of leukemia or liver cancer.

Provided is a use of the pharmaceutical composition in the preparation of a medicament for the treatment of tumor diseases.

Pharmacological tests have proved that a scutellarin amide derivative the present disclosure and a pharmaceutically acceptable salt thereof has a good effect against tumor cell proliferation, and can be used for the further preparation of anti-tumor drugs.

Further, the present disclosure provides a pharmaceutical composition comprising the scutellarin amide derivative and a pharmaceutically acceptable salt thereof and a pharmaceutically acceptable carrier.

The pharmaceutical composition has a good effect against tumor cell proliferation, and can be used for the preparation of anti-tumor drugs.

### DETAILED DESCRIPTION OF THE EMBODIMENTS

### Example 1

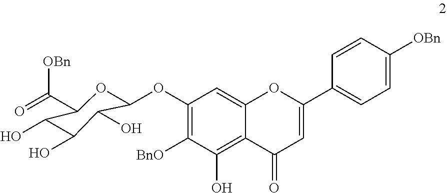 [SMILES: O=C(OCc1ccccc1)C1OC(Oc2cc3oc(-c4ccc(OCc5ccccc5)cc4)cc(=O)c3c(O)c2OCc2ccccc2)C(O)C(O)C1O]

Scutellarin 1 (300 mg, 0.65 mmol) is dissolved in DMF (10 ml); benzyl bromide (0.38 ml, 3 mmol) and anhydrous potassium carbonate (414 mg, 3 mmol) are added, stirred and reacted at room temperature; the progress of the reaction is monitored by TCL; the reaction is terminated after 24 hours. The reaction solution is poured into 20 ml of an ice-water mixture, extracted with ethyl acetate (30 ml×3), washed with a salt-saturated aqueous solution, and dried over anhydrous sodium sulfate; ethyl acetate is recovered; and crude product 2 is obtained and separated through a silica gel column (dichloromethane:methanol=20:1) to give 315 mg of yellow solids, with a yield of 67.2%. ESI-MS m/z 733.2 [M+H]⁺. ¹H NMR (DMSO-d₆, 400 MHz) δ: 12.96 (s, 1H, 5-OH), 8.03 (d, 2H, J=9.0 Hz, H-2′,6′), 7.55 (d, 2H, Ar—H), 7.48 (d, 2H, Ar—H), 7.36 (m, 9H, Ar—H), 7.25 (d, 2H, Ar—H), 7.20 (d, 2H, J=9.0 Hz, H-3′,5′), 7.12 (s, 1H, H-8), 6.95 (s, 1H, H-3), 5.66 (d, 1H, H-1″), 5.57 (d, 1H, sugar proton), 5.43 (d, 1H, sugar proton), 5.37 (d, 1H, sugar proton), 5.24 (s, 2H, —CH₂—), 5.17 (dd, 2H, —CH₂—), 5.02 (dd, 2H, —CH₂—), 4.29 (d, 1H, H-5″), 3.53-3.40 (m, 3H, H-2″,3″,4″).

### Example 2

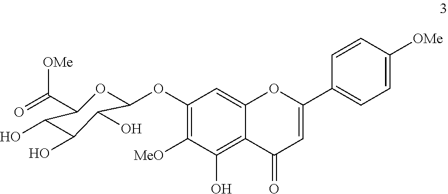 [SMILES: COC(=O)C1OC(Oc2cc3oc(-c4ccc(OC)cc4)cc(=O)c3c(O)c2OC)C(O)C(O)C1O]

Compound 3 is prepared by referring to the synthetic method of Example 1. A yellow powder is obtained with a yield of 41.9%, ESI-MS m/z 505.1 [M+H]⁺. ¹H NMR (DMSO-d₆, 400 MHz) δ: 12.94 (s, 1H, 5-OH), 8.06 (d, 2H, J=9.0 Hz, H-2′,6′), 7.15 (d, 2H, J=9.0 Hz, H-3′,5′), 7.09 (s, 1H, H-8), 6.96 (s, 1H, H-3), 5.61 (brs, 1H, H-1″), 5.50 (d, 1H, sugar proton), 5.37 (d, 1H, sugar proton), 5.33 (d, 1H, sugar proton), 4.21 (d, 1H, H-5″), 3.87 (s, 3H, —OCH₃), 3.76 (s, 3H, —OCH₃), 3.66 (s, 3H, —OCH₃), 3.48-3.35 (m, 3H, H-2″,3″,4″).

### Example 3

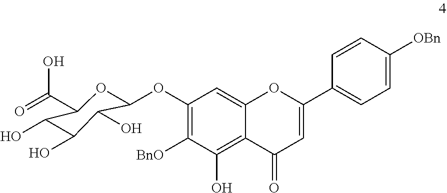 [SMILES: O=C(O)C1OC(Oc2cc3oc(-c4ccc(OCc5ccccc5)cc4)cc(=O)c3c(O)c2OCc2ccccc2)C(O)C(O)C1O]

Compound 2 (1 g, 1.37 mmol) in Example 1 is dissolved in methanol (80 ml); sodium methoxide (2-3 ml) is added dropwise and reacted at room temperature for 24 hours, which is concentrated, washed with acid, filtered with suction and dried to obtain 838 mg of yellow solids with a yield of 95.3%. ESI-MS m/z 643.2 [M+H]⁺. ¹H NMR (DMSO-d₆, 400 MHz) δ: 12.98 (s, 1H, 5-OH), 8.05 (d, 2H, J=8.5 Hz, H-2′,6′), 7.56 (d, 2H, Ar—H), 7.48 (d, 2H, Ar—H), 7.41 (t, 2H, Ar—H), 7.37 (q, 3H, Ar—H), 7.32 (t, 1H, Ar—H), 7.21 (d, 2H, J=8.7 Hz, H-3′,5′), 7.13 (s, 1H, H-8), 6.95 (s, 1H, H-3), 5.67 (d, 1H, H-1″), 5.40 (d, 2H, sugar proton), 5.22 (s, 2H, —CH₂—), 5.02 (dd, 2H, —CH₂—), 4.09 (d, 1H, H-5″), 3.48-3.42 (m, 3H, H-2″,3″,4″).

### Example 4

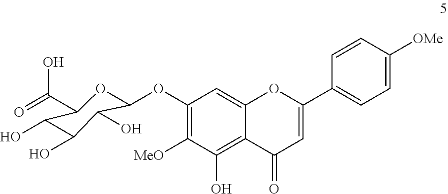 [SMILES: COc1ccc(-c2cc(=O)c3c(O)c(OC)c(OC4OC(C(=O)O)C(O)C(O)C4O)cc3o2)cc1]

Compound 5 is prepared by referring to the synthetic method of Example 3. A yellow powder is obtained with a yield of 94.1%, ESI-MS m/z 491.1 [M+H]⁺. ¹H NMR (DMSO-d₆, 400 MHz) δ: 12.94 (s, 1H, 5-OH), 8.06 (d, 2H, J=9.0 Hz, H-2′,6′), 7.15 (d, 2H, J=9.0 Hz, H-3′,5′), 7.09 (s, 1H, H-8), 6.96 (s, 1H, H-3), 5.61 (brs, 1H, H-1″), 5.50 (d, 1H, sugar proton), 5.37 (d, 1H, sugar proton), 5.33 (d, 1H, sugar proton), 4.21 (d, 1H, H-5″), 3.87 (s, 3H, —OCH₃), 3.76 (s, 3H, —OCH₃), 3.66 (s, 3H, —OCH₃), 3.48-3.35 (m, 3H, H-2″,3″,4″).

### Example 5

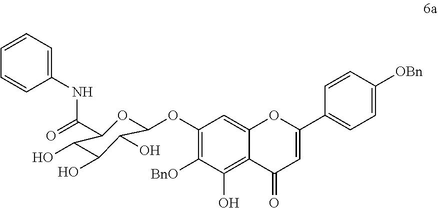 [SMILES: O=C(Nc1ccccc1)C1OC(Oc2cc3oc(-c4ccc(OCc5ccccc5)cc4)cc(=O)c3c(O)c2OCc2ccccc2)C(O)C(O)C1O]

Compound 4 (100 mg, 0.16 mmol) is dissolved in DMF (3 ml); HOBT (26 mg, 0.19 mmol) and EDCI (60 mg, 0.32 mmol) are added and stirred at room temperature for 15 minutes; aniline (57 μl, 0.62 mmol) is added and reacted at room temperature for 3 hours. The reaction is monitored by TLC and terminated when it is completed. The reaction solution is poured into 20 ml of distilled water, extracted three times with 20 ml of ethyl acetate, washed with a saturated salt solution, dried over anhydrous sodium sulfate, filtered, concentrated, and separated through a silica gel column (dichloromethane:methanol=20:1, V:V) to obtain 104 mg of white solids with a yield of 91%. ¹H NMR (DMSO-d₆, 400 MHz) δ: 12.95 (s, 1H, 5-OH), 10.21 (s, 1H, N—H), 8.09 (d, 2H, J=9.0 Hz, H-2′,6′), 7.67 (d, 2H, J=7.6 Hz, Ar—H), 7.57 (d, 2H, J=6.9 Hz, Ar—H), 7.50 (d, 2H, J=6.9 Hz, Ar—H), 7.42 (t, 2H, Ar—H), 7.34 (m, 5H, Ar—H), 7.27 (d, 2H, J=7.6 Hz, Ar—H), 7.23 (d, 2H, J=9.0 Hz, H-3′,5′), 7.14 (s, 1H, H-8), 6.95 (s, 1H, H-3), 5.66 (d, 1H, J=6.0 Hz, H-1″), 5.50 (d, 1H, J=5.7 Hz, sugar proton), 5.40 (d, 1H, J=5.5 Hz, sugar proton), 5.28 (s, 1H, sugar proton), 5.26 (s, 2H, —CH₂—), 5.09 (d, 1H, —CH₂—), 4.98 (d, 1H, —CH₂—), 4.10 (d, 1H, J=9.5 Hz, H-5″), 3.56-3.40 (m, 3H, H-2″, 3″, 4″).

### Example 6

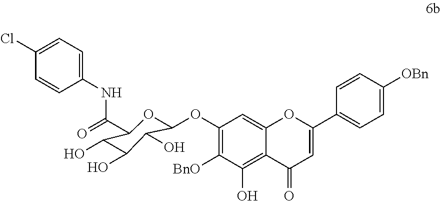 [SMILES: O=C(Nc1ccc(Cl)cc1)C1OC(Oc2cc3oc(-c4ccc(OCc5ccccc5)cc4)cc(=O)c3c(O)c2OCc2ccccc2)C(O)C(O)C1O]

Compound is prepare by referring to the synthetic method of Example 5. A yellow powder is obtained with a yield of 12.5%. ¹H NMR (DMSO-d₆, 600 MHz) δ (ppm): 13.00 (s, 1H, 5-OH), 8.11 (d, 2H, J=8.9 Hz, H-2′,6′), 7.51 (q, 5H, Ar—H), 7.37 (m, 9H, Ar—H), 7.20 (d, 2H, J=8.9 Hz, H-3′,5′), 7.03 (s, 1H, H-8), 6.99 (s, 1H, H-3), 6.47 (d, 1H, J=4.7 Hz, H-1″), 5.97 (d, 1H, J=7.1 Hz, sugar proton), 5.87 (s, 1H, sugar proton), 5.24 (s, 2H, —CH₂—), 4.94 (s, 2H, —CH₂—), 4.83 (t, 1H, H-5″), 4.49-4.19 (m, 3H, H-2″, 3″, 4″).

### Example 7

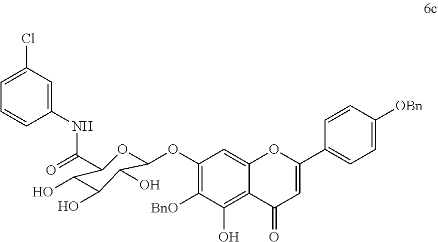 [SMILES: O=C(Nc1cccc(Cl)c1)C1OC(Oc2cc3oc(-c4ccc(OCc5ccccc5)cc4)cc(=O)c3c(O)c2OCc2ccccc2)C(O)C(O)C1O]

Compound 6c is prepared by referring to the synthetic method of Example 5. A yellow powder is obtained with a yield of 18.3%. ¹H NMR (DMSO-d₆, 600 MHz) δ (ppm): 12.95 (s, 1H, 5-OH), 10.35 (s, 1H, N—H), 8.08 (d, 2H, J=8.8 Hz, H-2′,6′), 7.86 (t, 1H, Ar—H), 7.57 (d, 2H, J=7.6 Hz, Ar—H), 7.49 (d, 2H, J=7.3 Hz, Ar—H), 7.42 (t, 2H, Ar—H), 7.37 (q, 4H, Ar—H), 7.32 (t, 1H, Ar—H), 7.29 (d, 1H, J=8.2 Hz, Ar—H), 7.23 (d, 2H, J=8.8 Hz, H-3′,5′), 7.12 (s, 1H, H-8), 7.10 (d, 1H, J=6.5 Hz, Ar—H), 6.94 (s, 1H, H-3), 5.65 (d, 1H, J=5.8 Hz, H-1″), 5.51 (d, 1H, J=5.6 Hz, sugar proton), 5.39 (d, 1H, J=5.3 Hz, sugar proton), 5.31 (d, 1H, J=7.6 Hz, sugar proton), 5.25 (s, 2H, —CH₂—), 5.10 (d, 1H, —CH₂—), 4.99 (d, 1H, —CH₂—), 4.10 (d, 1H, J=9.8 Hz, H-5″), 3.69-3.43 (m, 3H, H-2″, 3″, 4″).

### Example 8

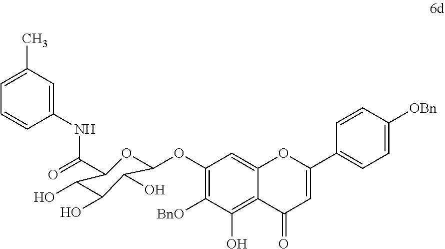 [SMILES: Cc1cccc(NC(=O)C2OC(Oc3cc4oc(-c5ccc(OCc6ccccc6)cc5)cc(=O)c4c(O)c3OCc3ccccc3)C(O)C(O)C2O)c1]

Compound 6d is prepared by referring to the synthetic method of Example 5. A yellow powder is obtained with a yield of 48.7%. ¹H NMR (DMSO-d₆, 400 MHz) δ (ppm): 12.95 (s, 1H, 5-OH), 10.11 (s, 1H, N—H), 8.11 (d, 2H, J=9.0 Hz, H-2′,6′), 7.57 (d, 2H, J=6.7 Hz, Ar—H), 7.49 (d, 2H, J=7.4 Hz, Ar—H), 7.42 (q, 4H, Ar—H), 7.35 (m, 5H, Ar—H), 7.23 (d, 2H, J=9.0 Hz, H-3′,5′), 7.14 (t, 2H, H-8, Ar—H), 6.95 (s, 1H, H-3), 6.85 (d, 1H, J=7.6 Hz, Ar—H), 5.65 (d, 1H, J=5.9 Hz, H-1″), 5.47 (d, 1H, J=5.6 Hz, sugar proton), 5.39 (d, 1H, J=5.3 Hz, sugar proton), 5.27 (d, 1H, J=7.9 Hz, sugar proton), 5.25 (s, 2H, —CH₂—), 5.09 (d, 1H, —CH₂—), 4.99 (d, 1H, —CH₂—), 4.10 (d, 1H, J=9.8 Hz, H-5″), 3.71-3.40 (m, 3H, H-2″, 3″, 4″), 2.20 (s, 3H, Ar—CH₃).

### Example 9

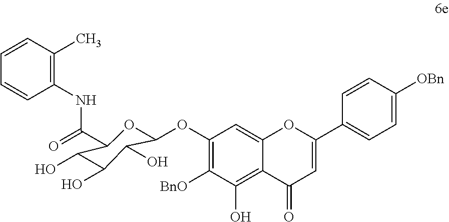 [SMILES: Cc1ccccc1NC(=O)C1OC(Oc2cc3oc(-c4ccc(OCc5ccccc5)cc4)cc(=O)c3c(O)c2OCc2ccccc2)C(O)C(O)C1O]

Compound 6e is prepared by referring to the synthetic method of Example 5. A yellow powder is obtained with a yield of 69.1%. ¹H NMR (DMSO-d₆, 400 MHz) δ (ppm): 12.97 (s, 1H, 5-OH), 9.41 (s, 1H, N—H), 8.08 (d, 2H, J=8.9 Hz, H-2′,6′), 7.57 (d, 2H, J=7.0 Hz, Ar—H), 7.49 (d, 2H, J=6.9 Hz, Ar—H), 7.41 (t, 3H, Ar—H), 7.36 (d, 3H, Ar—H), 7.21 (d, 3H, Ar—H, H-3′,5′), 7.14 (q, 3H, Ar—H), 7.05 (d, 1H, J=8.2 Hz, Ar—H), 6.98 (s, 1H, H-3), 5.65 (s, 1H, H-1″), 5.54 (s, 1H, sugar proton), 5.41 (d, 1H, J=6.2 Hz, sugar proton), 5.28 (d, 1H, J=7.5 Hz, sugar proton), 5.24 (s, 2H, —CH₂—), 5.10 (d, 2H, —CH₂—), 5.00 (d, 2H, —CH₂—), 4.26 (d, 1H, J=9.8 Hz, H-5″), 3.68-3.41 (m, 3H, H-2″, 3″, 4″), 2.17 (s, 3H, Ar—CH₃).

### Example 10

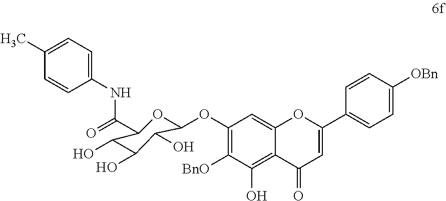 [SMILES: Cc1ccc(NC(=O)C2OC(Oc3cc4oc(-c5ccc(OCc6ccccc6)cc5)cc(=O)c4c(O)c3OCc3ccccc3)C(O)C(O)C2O)cc1]

Compound 6f is prepared by referring to the synthetic method of Example 5. A yellow powder is obtained with a yield of 67.5%. ¹H NMR (DMSO-d₆, 400 MHz) δ (ppm): 12.94 (s, 1H, 5-OH), 10.09 (s, 1H, N—H), 8.09 (d, 2H, J=9.0 Hz, H-2′,6′), 7.57 (d, 2H, J=7.3 Hz, Ar—H), 7.55 (d, 2H, J=8.5 Hz, Ar—H), 7.50 (d, 2H, J=7.2 Hz, Ar—H), 7.42 (t, 2H, Ar—H), 7.36 (m, 3H, Ar—H), 7.32 (d, 1H, J=7.2 Hz, Ar—H), 7.23 (d, 2H, J=9.0 Hz, H-3′,5′), 7.12 (s, 1H, H-8), 7.07 (d, 2H, J=8.4 Hz, Ar—H), 6.94 (s, 1H, H-3), 5.62 (brs, 1H, H-1″), 5.45 (s, 1H, sugar proton), 5.34 (s, 1H, sugar proton), 5.24 (s, 1H, sugar proton), 5.26 (s, 2H, —CH₂—), 5.09 (d, 1H, —CH₂—), 4.99 (d, 1H, —CH₂—), 4.08 (d, 1H, J=9.7 Hz, H-5″), 3.69-3.41 (m, 3H, H-2″, 3″, 4″), 2.22 (s, 3H, Ar—CH₃).

### Example 11

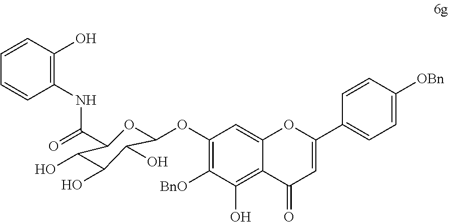 [SMILES: O=C(Nc1ccccc1O)C1OC(Oc2cc3oc(-c4ccc(OCc5ccccc5)cc4)cc(=O)c3c(O)c2OCc2ccccc2)C(O)C(O)C1O]

Compound g is prepare by re erring to the synthetic method of Example 5. A yellow powder is obtained with a yield of 19.6%. ¹H NMR (DMSO-d₆, 600 MHz) δ: 12.97 (s, 1H, 5-OH), 9.96 (s, 1H, N—H), 9.19 (s, 1H, Ar—OH), 8.05 (d, 2H, J=9.1 Hz, H-2′,6′), 7.57 (d, 2H, J=7.3 Hz, Ar—H), 7.48 (d, 2H, J=7.3 Hz, Ar—H), 7.41 (t, 2H, Ar—H), 7.35 (m, 5H, Ar—H), 7.22 (s, 2H, H-3′,5′), 7.20 (s, 1H, H-8), 6.94 (s, 1H, H-3), 6.90 (m, 1H, Ar—H), 6.85 (t, 1H, Ar—H), 6.75 (t, 1H, Ar—H), 5.64 (d, 1H, J=5.8 Hz, H-1″), 5.49 (d, 1H, J=5.2 Hz, sugar proton), 5.36 (d, 1H, J=5.5 Hz, sugar proton), 5.33 (d, 1H, J=7.7 Hz, sugar proton), 5.23 (s, 2H, —CH₂—), 5.10 (d, 1H, —CH₂—), 4.99 (d, 1H, —CH₂—), 4.38 (d, 1H, J=9.7 Hz, H-5″), 3.58-3.40 (m, 3H, H-2″, 3″, 4″).

### Example 12

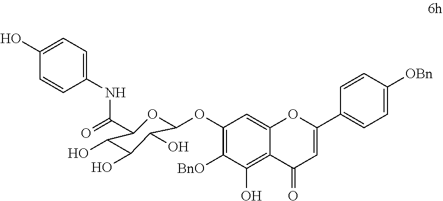 [SMILES: O=C(Nc1ccc(O)cc1)C1OC(Oc2cc3oc(-c4ccc(OCc5ccccc5)cc4)cc(=O)c3c(O)c2OCc2ccccc2)C(O)C(O)C1O]

Compound 6h is prepared by referring to the synthetic method of Example 5. A yellow powder is obtained with a yield of 22.1%. ¹H NMR (DMSO-d₆, 600 MHz) δ: 12.94 (s, 1H, 5-OH), 9.94 (s, 1H, N—H), 9.18 (s, 1H, Ar—OH), 8.09 (d, 2H, J=8.8 Hz, H-2′,6′), 7.57 (d, 2H, J=7.2 Hz, Ar—H), 7.50 (d, 2H, J=7.3 Hz, Ar—H), 7.45 (d, 2H, J=8.9 Hz, Ar—H), 7.42 (t, 2H, Ar—H), 7.37 (m, 3H, Ar—H), 7.31 (d, 1H, J=7.3 Hz, Ar—H), 7.23 (d, 2H, J=8.8 Hz, H-3′,5′), 7.12 (s, 1H, H-8), 6.95 (s, 1H, H-3), 6.66 (d, 2H, J=8.9 Hz, Ar—H), 5.61 (d, 1H, J=6.0 Hz, H-1″), 5.42 (d, 1H, J=5.5 Hz, sugar proton), 5.34 (d, 1H, J=5.4 Hz, sugar proton), 5.24 (s, 1H, sugar proton), 5.26 (s, 2H, —CH₂—), 5.10 (d, 1H, —CH₂—), 4.99 (d, 1H, —CH₂—), 4.05 (d, 1H, J=9.6 Hz, H-5″), 3.67-3.41 (m, 3H, H-2″, 3″, 4″).

### Example 13

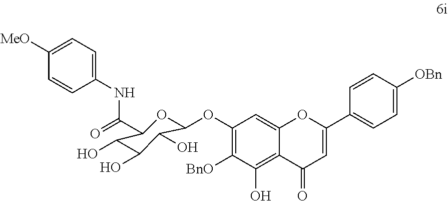 [SMILES: COc1ccc(NC(=O)C2OC(Oc3cc4oc(-c5ccc(OCc6ccccc6)cc5)cc(=O)c4c(O)c3OCc3ccccc3)C(O)C(O)C2O)cc1]

Compound 6i is prepared by referring to the synthetic method of Example 5. A yellow powder is obtained with a yield of 26.8%. ¹¹H NMR (DMSO-d₆, 400 MHz) δ: 12.96 (s, 1H, 5-OH), 10.05 (s, 1H, N—H), 8.09 (d, 2H, J=9.0 Hz, H-2′,6′), 7.57 (d, 2H, J=9.3 Hz, Ar—H), 7.49 (d, 2H, J=7.1 Hz, Ar—H), 7.43 (d, 2H, J=7.1 Hz, Ar—H), 7.36 (m, 4H, Ar—H), 7.32 (d, 2H, J=7.1 Hz, Ar—H), 7.23 (d, 2H, J=9.2 Hz, H-3′,5′), 7.12 (s, 1H, H-8), 6.95 (s, 1H, H-3), 6.85 (d, 2H, J=9.3 Hz, Ar—H), 5.65 (brs, 1H, H-1″), 5.47 (brs, 1H, sugar proton), 5.40 (brs, 1H, sugar proton), 5.28 (d, 1H, J=7.5 Hz, sugar proton), 5.25 (s, 2H, —CH₂—), 5.10 (d, 1H, —CH₂—), 4.99 (d, 1H, —CH₂—), 4.07 (d, 1H, J=9.8 Hz, H-5″), 3.69 (s, 3H, —OCH₃), 3.54-3.40 (m, 3H, H-2″, 3″, 4″).

### Example 14

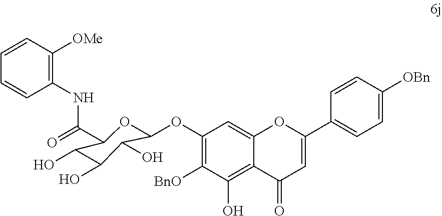 [SMILES: COc1ccccc1NC(=O)C1OC(Oc2cc3oc(-c4ccc(OCc5ccccc5)cc4)cc(=O)c3c(O)c2OCc2ccccc2)C(O)C(O)C1O]

Compound 6j is prepared by referring to the synthetic method of Example 5. A yellow powder is obtained with a yield of 23.1%. ¹H NMR (DMSO-d₆, 400 MHz) δ: 12.99 (s, 1H, 5-OH), 9.19 (s, 1H, N—H), 8.14 (d, 1H, J=7.0 Hz, Ar—H), 8.04 (d, 2H, J=9.0 Hz, H-2′,6′), 7.58 (d, 2H, J=7.0 Hz, Ar—H), 7.48 (d, 2H, J=7.0 Hz, Ar—H), 7.36 (m, 8H, Ar—H), 7.22 (s, 1H, H-8), 7.18 (d, 2H, J=9.2 Hz, H-3′,5′), 6.96 (s, 1H, H-3), 6.90 (t, 1H, Ar—H), 5.68 (d, 1H, J=5.6 Hz, H-1″), 5.57 (d, 1H, J=4.8 Hz, sugar proton), 5.40 (s, 1H, sugar proton), 5.34 (d, 1H, J=7.5 Hz, sugar proton), 5.25 (s, 2H, —CH₂—), 5.11 (d, 1H, —CH₂—), 5.00 (d, 1H, —CH₂—), 4.39 (d, 1H, J=9.8 Hz, H-5″), 3.70 (s, 3H, —OCH₃), 3.61-3.43 (m, 3H, H-2″, 3″, 4″).

### Example 15

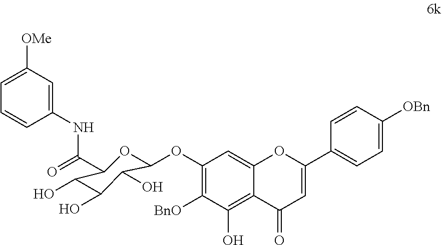 [SMILES: COc1cccc(NC(=O)C2OC(Oc3cc4oc(-c5ccc(OCc6ccccc6)cc5)cc(=O)c4c(O)c3OCc3ccccc3)C(O)C(O)C2O)c1]

Compound 6k is prepared by referring to the synthetic method of Example 5. A yellow powder is obtained with a yield of 25.3%. ¹H NMR (DMSO-d₆, 400 MHz) δ: 12.95 (s, 1H, 5-OH), 10.18 (s, 1H, N—H), 8.08 (d, 2H, J=9.0 Hz, H-2′,6′), 7.57 (d, 2H, J=7.0 Hz, Ar—H), 7.50 (d, 2H, J=7.0 Hz, Ar—H), 7.37 (m, 8H, Ar—H), 7.22 (d, 2H, J=9.2 Hz, H-3′,5′), 7.17 (d, 1H, J=8.10 Hz, Ar—H), 7.13 (s, 1H, H-8), 6.95 (s, 1H, H-3), 6.62 (m, 1H, Ar—H), 5.65 (s, 1H, H-1″), 5.50 (s, 1H, sugar proton), 5.38 (s, 1H, sugar proton), 5.28 (d, 1H, J=7.9 Hz, sugar proton), 5.24 (s, 2H, —CH₂—), 5.10 (d, 1H, —CH₂—), 4.99 (d, 1H, —CH₂—), 4.09 (d, 1H, J=9.8 Hz, H-5″), 3.68 (s, 3H, —OCH₃), 3.55-3.41 (m, 3H, H-2″, 3″, 4″).

### Example 16

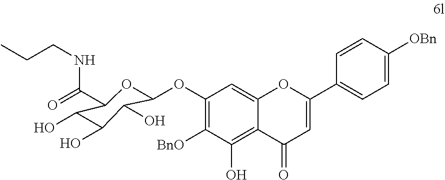 [SMILES: CCCNC(=O)C1OC(Oc2cc3oc(-c4ccc(OCc5ccccc5)cc4)cc(=O)c3c(O)c2OCc2ccccc2)C(O)C(O)C1O]

Compound 6l is prepared by referring to the synthetic method of Example 5. A yellow powder is obtained with a yield of 48.5%. ¹H NMR (DMSO-d₆, 400 MHz) δ: 12.96 (s, 1H, 5-OH), 8.08 (d, 2H, J=9.0 Hz, H-2′,6′), 8.01 (t, 1H, N—H), 7.57 (d, 2H, J=6.8 Hz, Ar—H), 7.49 (d, 2H, J=6.9 Hz, Ar—H), 7.41 (d, 2H, J=7.0 Hz, Ar—H), 7.35 (t, 4H, Ar—H), 7.21 (d, 2H, J=9.0 Hz, H-3′,5′), 7.07 (s, 1H, H-8), 6.96 (s, 1H, H-3), 5.59 (d, 1H, J=5.8 Hz, H-1″), 5.31 (d, 1H, J=5.3 Hz, sugar proton), 5.26 (d, 1H, J=5.0 Hz, sugar proton), 5.21 (d, 1H, J=7.7 Hz, sugar proton), 5.24 (s, 2H, —CH₂—), 5.09 (d, 1H, —CH₂—), 4.98 (d, 1H, —CH₂—), 3.94 (d, 1H, J=9.6 Hz, H-5″), 3.53-3.36 (m, 3H, H-2″, 3″, 4″), 3.03 (m, 2H, —CH₂—), 1.40 (m, 2H, —CH₂—), 0.78 (t, 3H, —CH₃).

### Example 17

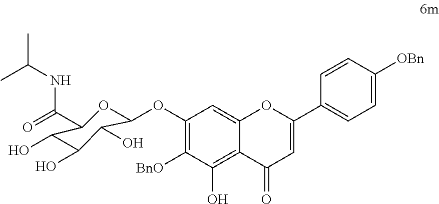 [SMILES: CC(C)NC(=O)C1OC(Oc2cc3oc(-c4ccc(OCc5ccccc5)cc4)cc(=O)c3c(O)c2OCc2ccccc2)C(O)C(O)C1O]

Compound 6m is prepared by referring to the synthetic method of Example 5. A yellow powder is obtained with a yield of 35.7%. ¹H NMR (DMSO-d₆, 400 MHz) δ: 12.94 (s, 1H, 5-OH), 8.09 (d, 2H, J=9.0 Hz, H-2′,6′), 7.95 (d, 1H, J=7.7 Hz, N—H), 7.56 (d, 2H, J=6.9 Hz, Ar—H), 7.48 (d, 2H, J=7.0 Hz, Ar—H), 7.41 (t, 2H, Ar—H), 7.36 (d, 3H, Ar—H), 7.32 (d, 1H, J=7.0 Hz, Ar—H), 7.20 (d, 2H, J=9.0 Hz, H-3′,5′), 7.09 (s, 1H, H-8), 6.96 (s, 1H, H-3), 5.59 (d, 1H, J=5.9 Hz, H-1″), 5.31 (d, 1H, J=5.2 Hz, sugar proton), 5.27 (d, 1H, J=5.1 Hz, sugar proton), 5.17 (d, 1H, J=7.7 Hz, sugar proton), 5.24 (s, 2H, —CH₂—), 5.09 (d, 1H, —CH₂—), 4.98 (d, 1H, —CH₂—), 3.87 (d, 1H, J=9.4 Hz, H-5″), 3.82 (q, 1H, —CH—), 3.58-3.43 (m, 3H, H-2″, 3″, 4″), 1.07 (d, 3H, —CH₃), 1.02 (d, 3H, —CH₃).

### Example 18

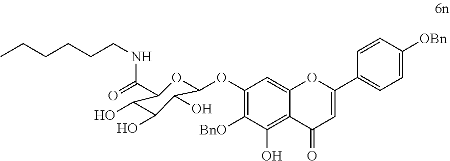 [SMILES: CCCCCCNC(=O)C1OC(Oc2cc3oc(-c4ccc(OCc5ccccc5)cc4)cc(=O)c3c(O)c2OCc2ccccc2)C(O)C(O)C1O]

Compound n is prepare by referring to the synthetic method of Example 5. A yellow powder is obtained with a yield of 38.9%. ¹H NMR (DMSO-d₆, 400 MHz) δ: 12.95 (s, 1H, 5-OH), 8.10 (d, 2H, J=8.9 Hz, H-2′,6′), 8.00 (t, 1H, N—H), 7.56 (d, 2H, J=6.9 Hz, Ar—H), 7.49 (d, 2H, J=7.1 Hz, Ar—H), 7.42 (t, 2H, Ar—H), 7.36 (d, 3H, Ar—H), 7.32 (d, 1H, J=7.2 Hz, Ar—H), 7.21 (d, 2H, J=9.0 Hz, H-3′,5′), 7.07 (s, 1H, H-8), 6.98 (s, 1H, H-3), 5.60 (brs, 1H, H-1″), 5.20 (d, 1H, J=7.8 Hz, sugar proton), 5.24 (s, 2H, —CH₂—), 5.09 (d, 1H, —CH₂—), 4.98 (d, 1H, —CH₂—), 3.92 (d, 1H, J=9.6 Hz, H-5″), 3.54-3.43 (m, 4H, H-2″, 3″, 4″), 3.06 (m, 2H, —CH₂—), 1.35 (t, 2H, —CH₂—), 1.15 (t, 2H, —CH₂—), 1.04 (t, 4H, —CH₂—×2), 0.65 (t, 3H, —CH₃).

### Example 19

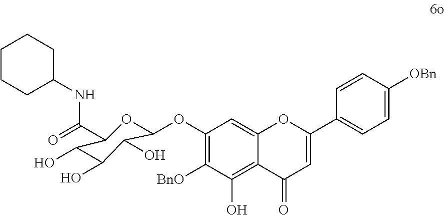 [SMILES: O=C(NC1CCCCC1)C1OC(Oc2cc3oc(-c4ccc(OCc5ccccc5)cc4)cc(=O)c3c(O)c2OCc2ccccc2)C(O)C(O)C1O]

Compound 6o is prepared by referring to the synthetic method of Example 5. A yellow powder is obtained with a yield of 38.9%. ¹H NMR (DMSO-d₆, 400 MHz) δ: 12.94 (s, 1H, 5-OH), 8.10 (d, 2H, J=8.9 Hz, H-2′,6′), 7.94 (d, 1H, J=8.1 Hz, N—H), 7.56 (d, 2H, J=6.9 Hz, Ar—H), 7.48 (d, 2H, J=7.0 Hz, Ar—H), 7.41 (t, 3H, Ar—H), 7.36 (d, 2H, J=7.7 Hz, Ar—H), 7.32 (d, 1H, J=7.1 Hz, Ar—H), 7.19 (d, 2H, J=9.0 Hz, H-3′,5′), 7.11 (s, 1H, H-8), 6.97 (s, 1H, H-3), 5.59 (brs, 1H, H-1″), 5.28 (t, 4H, —CH₂, sugar proton×2), 5.14 (d, 1H, J=7.8 Hz, sugar proton), 5.09 (d, 1H, —CH₂—), 4.98 (d, 1H, —CH₂—), 3.90 (d, 1H, J=9.7 Hz, H-5″), 3.57-3.43 (m, 4H, H-2″, 3″, 4″), 1.58 (m, 6H, —CH₂—×3), 1.19 (q, 4H, —CH₂—×2).

### Example 20

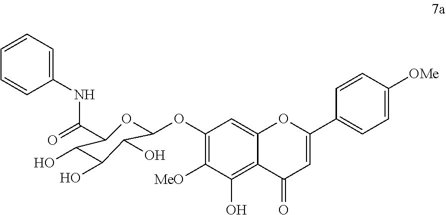 [SMILES: COc1ccc(-c2cc(=O)c3c(O)c(OC)c(OC4OC(C(=O)Nc5ccccc5)C(O)C(O)C4O)cc3o2)cc1]

Compound 7a is prepared by referring to the synthetic method of Example 5. A yellow powder is obtained with a yield of 61.9%. ¹H NMR (DMSO-d₆, 400 MHz) δ: 12.92 (s, 1H, 5-OH), 10.22 (s, 1H, N—H), 8.09 (d, 2H, J=8.9 Hz, H-2′,6′), 7.67 (d, 2H, J=7.8 Hz, Ar—H), 7.29 (d, 2H, J=7.8 Hz, Ar—H), 7.15 (d, 2H, J=9.0 Hz, H-3′,5′), 7.11 (s, 1H, H-8), 7.06 (t, 1H, Ar—H), 6.96 (s, 1H, H-3), 5.61 (d, 1H, J=5.7 Hz, H-1″), 5.49 (d, 1H, J=5.6 Hz, sugar proton), 5.36 (d, 1H, J=5.2 Hz, sugar proton), 5.21 (d, 1H, J=7.6 Hz, sugar proton), 4.08 (d, 1H, J=9.6 Hz, H-5″), 3.89 (s, 3H, —OCH₃), 3.77 (s, 3H, —OCH₃), 3.50-3.39 (m, 3H, H-2″, 3″, 4″).

### Example 21

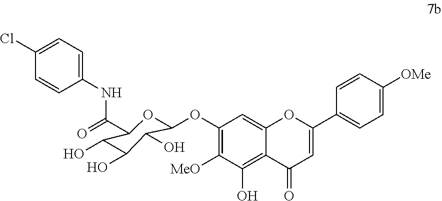 [SMILES: COc1ccc(-c2cc(=O)c3c(O)c(OC)c(OC4OC(C(=O)Nc5ccc(Cl)cc5)C(O)C(O)C4O)cc3o2)cc1]

Compound is prepare by referring to the synthetic method of Example 5. A yellow powder is obtained with a yield of 26.4%. ¹H NMR (DMSO-d₆, 400 MHz) δ: 12.92 (s, 1H, 5-OH), 10.32 (s, 1H, N—H), 8.08 (d, 2H, J=8.8 Hz, H-2′,6′), 7.70 (d, 2H, J=8.8 Hz, Ar—H), 7.34 (d, 2H, J=9.1 Hz, Ar—H), 7.15 (d, 2H, J=9.1 Hz, H-3′,5′), 7.09 (s, 1H, H-8), 6.95 (s, 1H, H-3), 5.60 (d, 1H, J=5.8 Hz, H-1″), 5.49 (d, 1H, J=5.6 Hz, sugar proton), 5.35 (d, 1H, J=5.1 Hz, sugar proton), 5.24 (d, 1H, J=7.5 Hz, sugar proton), 4.07 (d, 1H, J=9.7 Hz, H-5″), 3.89 (s, 3H, —OCH₃), 3.77 (s, 3H, —OCH₃), 3.48-3.37 (m, 3H, H-2″, 3″, 4″).

### Example 22

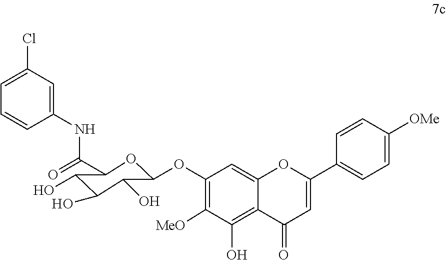 [SMILES: COc1ccc(-c2cc(=O)c3c(O)c(OC)c(OC4OC(C(=O)Nc5cccc(Cl)c5)C(O)C(O)C4O)cc3o2)cc1]

Compound 7c is prepared by referring to the synthetic method of Example 5. A yellow powder is obtained with a yield of 29.2%. ¹H NMR (DMSO-d₆, 400 MHz) δ: 12.92 (s, 1H, 5-OH), 10.39 (s, 1H, N—H), 8.08 (d, 2H, J=8.7 Hz, H-2′,6′), 7.85 (s, 1H, Ar—H), 7.57 (d, 1H, Ar—H), 7.32 (t, 1H, Ar—H), 7.14 (d, 2H, J=8.7 Hz, H-3′,5′), 7.12 (d, 1H, Ar—H), 7.10 (s, 1H, H-8), 6.95 (s, 1H, H-3), 5.61 (d, 1H, J=5.8 Hz, H-1″), 5.50 (d, 1H, J=5.4 Hz, sugar proton), 5.36 (d, 1H, J=5.0 Hz, sugar proton), 5.24 (d, 1H, J=7.5 Hz, sugar proton), 4.08 (d, 1H, J=9.8 Hz, H-5″), 3.88 (s, 3H, —OCH₃), 3.78 (s, 3H, —OCH₃), 3.51-3.38 (m, 3H, H-2″, 3″, 4″).

### Example 23

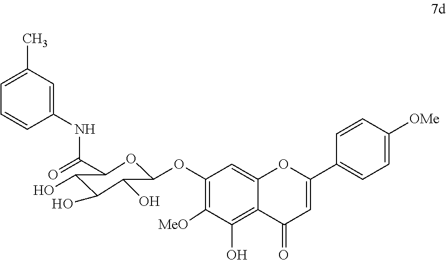 [SMILES: COc1ccc(-c2cc(=O)c3c(O)c(OC)c(OC4OC(C(=O)Nc5cccc(C)c5)C(O)C(O)C4O)cc3o2)cc1]

Compound 7d is prepared by referring to the synthetic method of Example 5. A yellow powder is obtained with a yield of 83.3%. ¹H NMR (DMSO-d₆, 400 MHz) δ: 12.91 (s, 1H, 5-OH), 10.14 (s, 1H, N—H), 8.11 (d, 2H, J=9.0 Hz, H-2′,6′), 7.16 (q, 3H, Ar—H), 7.12 (d, 3H, J=9.0 Hz, H-3′,5′, H-8), 6.96 (s, 1H, H-3), 6.88 (d, 1H, J=7.6 Hz, Ar—H), 5.61 (d, 1H, J=5.8 Hz, H-1″), 5.47 (d, 1H, J=5.5 Hz, sugar proton), 5.35 (d, 1H, J=5.1 Hz, sugar proton), 5.20 (d, 1H, J=7.5 Hz, sugar proton), 4.08 (d, 1H, J=9.6 Hz, H-5″), 3.88 (s, 3H, —OCH₃), 3.77 (s, 3H, —OCH₃), 3.49-3.37 (m, 3H, H-2″, 3″, 4″), 2.21 (s, 3H, Ar—CH₃).

### Example 24

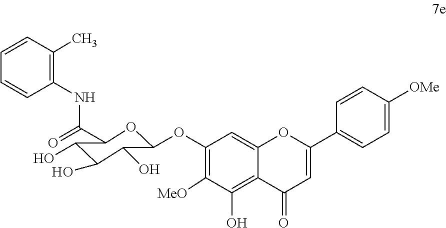 [SMILES: COc1ccc(-c2cc(=O)c3c(O)c(OC)c(OC4OC(C(=O)Nc5ccccc5C)C(O)C(O)C4O)cc3o2)cc1]

Compound 7e is prepared by referring to the synthetic method of Example 5. A yellow powder is obtained with a yield of 33.1%. ¹H NMR (DMSO-d₆, 400 MHz) δ: 12.95 (s, 1H, 5-OH), 9.43 (s, 1H, N—H), 8.08 (d, 2H, J=8.7 Hz, H-2′,6′), 7.18 (t, 2H, Ar—H), 7.12 (d, 3H, J=9.0 Hz, H-3′,5′, H-8), 7.05 (d, 1H, Ar—H), 6.98 (s, 1H, H-3), 6.87 (q, 1H, Ar—H), 5.60 (d, 1H, J=3.7 Hz, H-1″), 5.53 (s, 1H, sugar proton), 5.36 (s, 1H, sugar proton), 5.20 (d, 1H, J=7.2 Hz, sugar proton), 4.24 (d, 1H, J=9.6 Hz, H-5″), 3.86 (s, 3H, —OCH₃), 3.78 (s, 3H, —OCH₃), 3.50-3.39 (m, 3H, H-2″, 3″, 4″), 2.19 (s, 3H, Ar—CH₃).

### Example 25

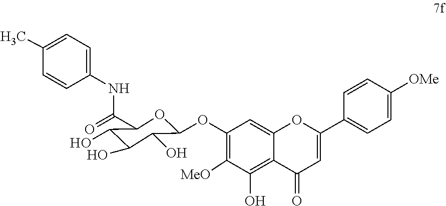 [SMILES: COc1ccc(-c2cc(=O)c3c(O)c(OC)c(OC4OC(C(=O)Nc5ccc(C)cc5)C(O)C(O)C4O)cc3o2)cc1]

Compound 7f is prepared by referring to the synthetic method of Example 5. A yellow powder is obtained with a yield of 51.9%. ¹H NMR (DMSO-d₆, 400 MHz) δ: 12.92 (s, 1H, 5-OH), 10.14 (s, 1H, N—H), 8.10 (d, 2H, J=9.0 Hz, H-2′,6′), 7.56 (d, 2H, J=8.4 Hz, Ar—H), 7.12 (d, 2H, J=9.0 Hz, H-3′,5′), 7.10 (s, 1H, H-8), 7.08 (d, 2H, J=8.4 Hz, Ar—H), 6.97 (s, 1H, H-3), 6.88 (d, 1H, J=7.6 Hz, Ar—H), 5.61 (d, 1H, J=5.7 Hz, H-1″), 5.48 (d, 1H, J=5.4 Hz, sugar proton), 5.36 (d, 1H, J=5.2 Hz, sugar proton), 5.20 (d, 1H, J=7.5 Hz, sugar proton), 4.06 (d, 1H, J=9.6 Hz, H-5″), 3.90 (s, 3H, —OCH₃), 3.77 (s, 3H, —OCH₃), 3.48-3.38 (m, 3H, H-2″, 3″, 4″), 2.22 (s, 3H, Ar—CH₃).

### Example 26

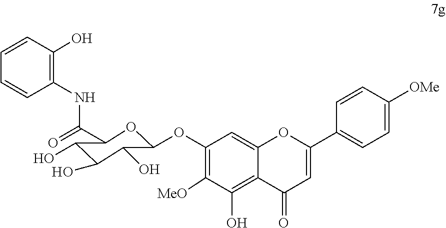 [SMILES: COc1ccc(-c2cc(=O)c3c(O)c(OC)c(OC4OC(C(=O)Nc5ccccc5O)C(O)C(O)C4O)cc3o2)cc1]

Compound 7g is prepared by referring to the synthetic method of Example 5. A yellow powder is obtained with a yield of 32.9%. ¹H NMR (DMSO-d₆, 400 MHz) δ: 12.94 (s, 1H, 5-OH), 10.06 (s, 1H, N—H), 9.20 (s, 1H, Ar—OH), 8.05 (d, 2H, J=8.8 Hz, H-2′,6′), 7.19 (s, 1H, H-8), 7.13 (d, 2H, J=9.0 Hz, H-3′,5′), 6.95 (s, 1H, H-3), 6.88 (m, 2H, Ar—H), 6.75 (m, 1H, Ar—H), 5.61 (brs, 1H, H-1″), 5.48 (brs, 1H, sugar proton), 5.33 (d, 1H, J=4.7 Hz, sugar proton), 5.28 (d, 1H, J=7.3 Hz, sugar proton), 4.36 (d, 1H, J=9.7 Hz, H-5″), 3.86 (s, 3H, —OCH₃), 3.79 (s, 3H, —OCH₃), 3.57-3.51 (m, 3H, H-2″, 3″, 4″).

### Example 27

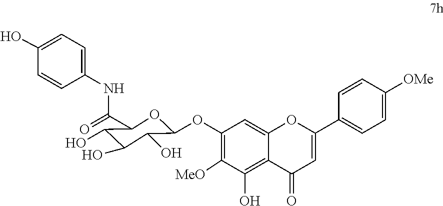 [SMILES: COc1ccc(-c2cc(=O)c3c(O)c(OC)c(OC4OC(C(=O)Nc5ccc(O)cc5)C(O)C(O)C4O)cc3o2)cc1]

Compound 7h is prepared by referring to the synthetic method of Example 5. A yellow powder is obtained with a yield of 27.2%. ¹H NMR (DMSO-d₆, 400 MHz) δ: 12.91 (s, 1H, 5-OH), 10.01 (s, 1H, N—H), 9.22 (s, 1H, Ar—OH), 8.09 (d, 2H, J=8.9 Hz, H-2′,6′), 7.47 (d, 2H, J=8.9 Hz, Ar—H), 7.14 (d, 2H, J=8.9 Hz, H-3′,5′), 7.11 (s, 1H, H-8), 6.95 (s, 1H, H-3), 6.66 (d, 2H, J=8.8 Hz, Ar—H), 5.59 (brs, 1H, H-1″), 5.43 (brs, 1H, sugar proton), 5.31 (d, 1H, J=4.8 Hz, sugar proton), 5.18 (d, 1H, J=7.5 Hz, sugar proton), 4.05 (d, 1H, J=9.7 Hz, H-5″), 3.89 (s, 3H, —OCH₃), 3.77 (s, 3H, —OCH₃), 3.66-3.58 (m, 3H, H-2″, 3″, 4″).

### Example 28

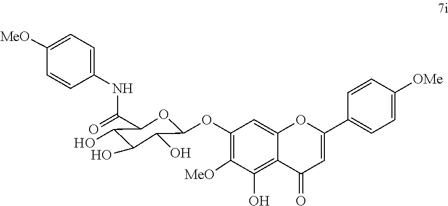 [SMILES: COc1ccc(NC(=O)C2OC(Oc3cc4oc(-c5ccc(OC)cc5)cc(=O)c4c(O)c3OC)C(O)C(O)C2O)cc1]

Compound 7i is prepared by referring to the synthetic method of Example 5. A yellow powder is obtained with a yield of 19.6%. ¹H NMR (DMSO-d₆, 400 MHz) δ: 12.92 (s, 1H, 5-OH), 10.07 (s, 1H, N—H), 8.07 (d, 2H, J=8.7 Hz, H-2′,6′), 7.58 (d, 2H, J=9.0 Hz, Ar—H), 7.13 (d, 2H, J=9.0 Hz, H-3′,5′), 7.09 (s, 1H, H-8), 6.94 (s, 1H, H-3), 6.85 (d, 2H, J=9.2 Hz, Ar—H), 5.59 (d, 1H, J=5.4 Hz, H-1″), 5.45 (d, 1H, J=5.2 Hz, sugar proton), 5.33 (d, 1H, J=5.0 Hz, sugar proton), 5.22 (d, 1H, J=7.4 Hz, sugar proton), 4.05 (d, 1H, J=9.7 Hz, H-5″), 3.88 (s, 3H, —OCH₃), 3.78 (s, 3H, —OCH₃), 3.69 (s, 3H, —OCH₃), 3.50-3.39 (m, 3H, H-2″, 3″, 4″).

### Example 29

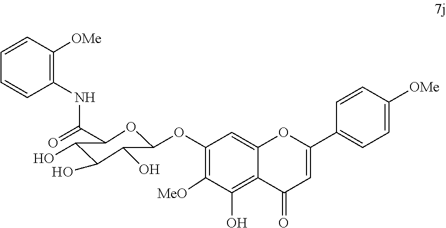 [SMILES: COc1ccc(-c2cc(=O)c3c(O)c(OC)c(OC4OC(C(=O)Nc5ccccc5OC)C(O)C(O)C4O)cc3o2)cc1]

Compound 7j is prepared by referring to the synthetic method of Example 5. A yellow powder is obtained with a yield of 29.7%. ¹H NMR (DMSO-d₆, 400 MHz) δ: 12.95 (s, 1H, 5-OH), 9.20 (s, 1H, N—H), 8.14 (d, 1H, J=7.6 Hz, Ar—H), 8.03 (d, 2H, J=8.7 Hz, H-2′,6′), 7.18 (s, 1H, H-8), 7.09 (d, 2H, J=9.0 Hz, H-3′,5′), 7.02 (t, 2H, Ar—H), 6.94 (s, 1H, H-3), 6.89 (t, 1H, Ar—H), 5.64 (d, 1H, J=5.1 Hz, H-1″), 5.55 (d, 1H, J=5.1 Hz, sugar proton), 5.37 (d, 1H, J=4.7 Hz, sugar proton), 5.28 (d, 1H, J=7.0 Hz, sugar proton), 4.38 (d, 1H, J=9.7 Hz, H-5″), 3.85 (s, 3H, —OCH₃), 3.80 (s, 3H, —OCH₃), 3.75 (s, 3H, —OCH₃), 3.60-3.41 (m, 3H, H-2″, 3″, 4″).

### Example 30

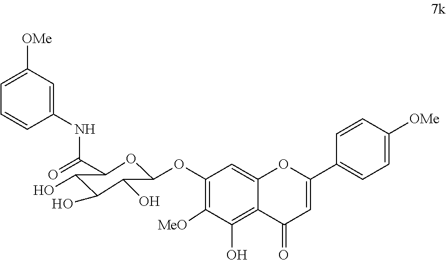 [SMILES: COc1ccc(-c2cc(=O)c3c(O)c(OC)c(OC4OC(C(=O)Nc5cccc(OC)c5)C(O)C(O)C4O)cc3o2)cc1]

Compound 7k is prepared by referring to the synthetic method of Example 5. A yellow powder is obtained with a yield of 33.6%. ¹H NMR (DMSO-d₆, 400 MHz) δ: 12.91 (s, 1H, 5-OH), 10.21 (s, 1H, N—H), 8.04 (d, 2H, J=8.7 Hz, H-2′,6′), 7.36 (t, 1H, Ar—H), 7.21 (m, 2H, Ar—H), 7.10 (d, 2H, J=9.0 Hz, H-3′,5′), 7.08 (s, 1H, H-8), 6.91 (s, 1H, H-3), 6.62 (m, 1H, Ar—H), 5.63 (d, 1H, J=5.2 Hz, H-1″), 5.50 (d, 1H, J=5.2 Hz, sugar proton), 5.38 (d, 1H, J=4.4 Hz, sugar proton), 5.23 (d, 1H, J=7.4 Hz, sugar proton), 4.09 (d, 1H, J=9.7 Hz, H-5″), 3.86 (s, 3H, —OCH₃), 3.78 (s, 3H, —OCH₃), 3.68 (s, 3H, —OCH₃), 3.49-3.44 (m, 3H, H-2″, 3″, 4″).

### Example 31

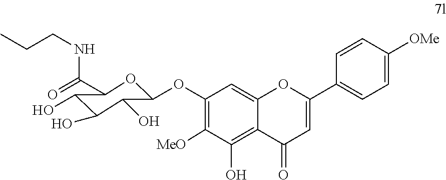 [SMILES: CCCNC(=O)C1OC(Oc2cc3oc(-c4ccc(OC)cc4)cc(=O)c3c(O)c2OC)C(O)C(O)C1O]

Compound 7l is prepared by referring to the synthetic method of Example 5. A yellow powder is obtained with a yield of 53.3%. ¹H NMR (DMSO-d₆, 400 MHz) δ: 12.92 (s, 1H, 5-OH), 8.08 (d, 2H, J=8.9 Hz, H-2′,6′), 8.01 (t, 1H, N—H), 7.13 (d, 2H, J=8.9 Hz, H-3′,5′), 7.04 (s, 1H, H-8), 6.97 (s, 1H, H-3), 5.52 (brs, 1H, H-1″), 5.22 (brs, 1H, sugar proton), 5.15 (d, 1H, J=7.5 Hz, sugar proton), 3.91 (d, 1H, J=9.7 Hz, H-5″), 3.87 (s, 3H, —OCH₃), 3.77 (s, 3H, —OCH₃), 3.52-3.42 (m, 3H, H-2″, 3″, 4″), 3.04 (m, 2H, —CH₂—), 1.41 (m, 2H, —CH₂—), 0.79 (t, 3H, —CH₃).

### Example 32

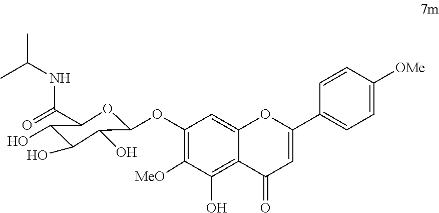 [SMILES: COc1ccc(-c2cc(=O)c3c(O)c(OC)c(OC4OC(C(=O)NC(C)C)C(O)C(O)C4O)cc3o2)cc1]

Compound 7m is prepared by referring to the synthetic method of Example 5. A yellow powder is obtained with a yield of 48.6%. ¹H NMR (DMSO-d₆, 400 MHz) δ: 12.91 (s, 1H, 5-OH), 8.09 (d, 2H, J=8.9 Hz, H-2′,6′), 7.94 (q, 1H, N—H), 7.12 (d, 2H, J=8.9 Hz, H-3′,5′), 7.05 (s, 1H, H-8), 6.96 (s, 1H, H-3), 5.52 (brs, 1H, H-1″), 5.24 (brs, 1H, sugar proton), 5.10 (d, 1H, J=7.6 Hz, sugar proton), 3.87 (s, 3H, —OCH₃), 3.82 (d, 1H, J=7.1 Hz, H-5″), 3.77 (s, 3H, —OCH₃), 3.52 (t, 1H, —CH—), 3.39-3.37 (t, 3H, H-2″, 3″, 4″), 1.08 (d, 3H, —CH₃), 1.03 (d, 3H, —CH₃).

### Example 33

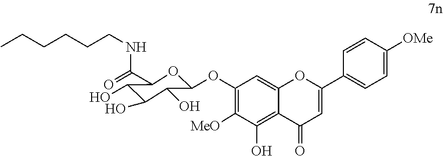 [SMILES: CCCCCCNC(=O)C1OC(Oc2cc3oc(-c4ccc(OC)cc4)cc(=O)c3c(O)c2OC)C(O)C(O)C1O]

Compound 7n is prepared by referring to the synthetic method of Example 5. A yellow powder is obtained with a yield of 36.1%. ¹H NMR (DMSO-d₆, 400 MHz) δ: 12.91 (s, 1H, 5-OH), 8.08 (d, 2H, J=8.7 Hz, H-2′,6′), 8.00 (s, 1H, N—H), 7.11 (d, 2H, J=8.7 Hz, H-3′,5′), 7.03 (s, 1H, H-8), 6.96 (s, 1H, H-3), 5.54 (d, 1H, J=5.4 Hz, H-1″), 5.25 (d, 1H, J=5.1 Hz, sugar proton), 5.22 (d, 1H, J=5.0 Hz, sugar proton), 5.15 (d, 1H, J=7.3 Hz, sugar proton), 3.91 (d, 1H, J=9.7 Hz, H-5″), 3.86 (s, 3H, —OCH₃), 3.77 (s, 3H, —OCH₃), 3.52-3.37 (m, 3H, H-2″, 3″, 4″), 3.06 (m, 2H, —CH₂), 1.36-1.05 (m, 8H, —CH₂—×4), 0.66 (t, 3H, —CH₃).

### Example 34

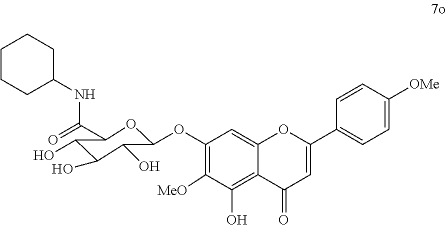 [SMILES: COc1ccc(-c2cc(=O)c3c(O)c(OC)c(OC4OC(C(=O)NC5CCCCC5)C(O)C(O)C4O)cc3o2)cc1]

Compound 7o is prepared by referring to the synthetic method of Example 5. A yellow powder is obtained with a yield of 13.1%. ¹H NMR (DMSO-d₆, 400 MHz) δ: 12.90 (s, 1H, 5-OH), 8.11 (d, 2H, J=9.0 Hz, H-2′,6′), 7.98 (d, 1H, N—H), 7.11 (d, 2H, J=9.0 Hz, H-3′,5′), 7.08 (s, 1H, H-8), 6.97 (s, 1H, H-3), 5.53 (s, 1H, H-1″), 5.25 (s, 2H, sugar proton), 5.08 (d, 1H, J=7.4 Hz, sugar proton), 3.88 (s, 1H, H-5″), 3.86 (s, 3H, —OCH₃), 3.77 (s, 3H, —OCH₃), 3.55-3.41 (m, 3H, H-2″, 3″, 4″), 1.73-1.22 (m, 11H, —CH₂—×5, —CH₃).

The following are the pharmacological test results of some compounds of the present disclosure:

Experimental Equipments and Reagents

Equipments: ultra-clean workbench (Suzhou Antai AirTech Co., Ltd.)

Constant temperature incubator (Thermo Electron Corporation)

Microplate reader (Bio-Rad Corporation)

Inverted biological microscope (Chongqing Optical Instrument Factory)

Reagents: cell culture medium RPMI-1640, DMEM (high glucose) (Gibco Corporation)

Fetal bovine serum (Hangzhou Sijiqing Co., Ltd.)

CCK-8 (Product of Biosharp Corporation)

Trypan blue (Product of Solarbio Corporation)

DMSO (Sigma Corporation)

Cell lines: human promyelocytic leukemia cell line HL-60, human acute leukemia mononuclear macrophage cell line THP-1, human liver cancer cell line HepG-2

Experimental Methods

Test Method for Cell Inhibitory Activity

Cells are routinely cultured in an incubator at 37° C. and 5% CO₂ saturated humidity. The culture medium is RPMI1640 cell culture medium containing 10% of heat-inactivated fetal bovine serum, 100 U/mL of penicillin and 100 U/mL of streptomycin. The culture medium is replaced after 48 hours. After the cells adhere to the wall, they are digested with 0.25% trypsin and passage. The experimental cells are all in the logarithmic growth phase, and the trypan blue exclusion method shows that the cell viability is >95%.

A vial of cells in the logarithmic growth phase and in good condition is digested with a digestion solution (0.125% trypsin+0.01% EDTA); cell count is 2 to 4×10⁴ cell/mL; a cell suspension is made and inoculated on a 96-well plate, 100 μL/well, and cultured in a constant temperature CO₂ incubator for 24 hours. The medium is replaced; the test drug is added, 100 μL/well, and cultured for 72 hours. CCK-8 is added to a 96-well plate, 50 μL/well, and incubated in an incubator for 4 hours. The supernatant is aspirated; DMSO is added, 200 μL/well, and shaken on a plate shaker for 10 minutes. The test drug is investigated at six concentrations from 0.001 to 100 μM in tenfold increase in concentration; the absorbance of each well is measured with an enzyme-linked immunosorbent monitor at a wavelength of 450 nm; and the cell inhibition rate at each concentration is calculated, respectively.

Calculation Method for Inhibition Rate:

$$Cell inhibition = \frac{\begin{matrix}Relative OD value of negative control wells - \\ relative OD value of drug sensitive wells\end{matrix}}{Relative OD value of negative control wells} \times 100 \%$$

Test results

TABLE 1
The IC₅₀ values (μM) of the anti-proliferative activity
on three human cancer cell lines in the examples

| | IC₅₀ (μM) | | |
|---|---|---|---|
| Samples | HL-60 | THP-1 | HepG-2 |
| Scutellarin | 22.70 ± 1.97 | 48.51 ± 2.21 | 56.09 ± 1.23 |
| Example 5 | 20.00 ± 0.19 | 35.37 ± 0.23 | 45.69 ± 2.15 |
| Example 6 | 75.88 ± 0.31 | 81.36 ± 0.35 | 51.65 ± 0.97 |
| Example 7 | 15.43 ± 0.11 | 13.67 ± 0.13 | 11.21 ± 1.07 |
| Example 8 | 11.84 ± 0.34 | 16.08 ± 0.57 | 12.03 ± 3.52 |
| Example 9 | 27.53 ± 0.15 | 26.77 ± 0.32 | 19.45 ± 1.11 |
| Example 10 | 12.99 ± 0.28 | 5.75 ± 0.36 | 7.23 ± 0.31 |
| Example 11 | 2.91 ± 0.69 | 6.11 ± 0.97 | 5.11 ± 0.43 |
| Example 12 | 58.86 ± 0.97 | 33.94 ± 0.78 | 36.11 ± 0.67 |
| Example 13 | 20.92 ± 0.46 | 26.28 ± 0.94 | 23.94 ± 0.88 |
| Example 14 | 1.70 ± 0.11 | 12.48 ± 0.35 | 6.23 ± 2.03 |
| Example 15 | 4.28 ± 0.31 | 31.62 ± 0.25 | 29.21 ± 0.77 |
| Example 16 | 30.61 ± 0.57 | 22.44 ± 0.38 | 21.03 ± 0.81 |
| Example 17 | 54.10 ± 1.21 | 37.29 ± 0.93 | 39.45 ± 1.03 |
| Example 18 | 59.05 ± 0.91 | 35.76 ± 1.24 | 46.21 ± 2.03 |
| Example 19 | 58.82 ± 2.31 | 16.77 ± 0.19 | 17.59 ± 1.66 |
| Example 20 | 85.23 ± 0.75 | 63.12 ± 0.46 | 16.77 ± 0.19 |
| Example 21 | 4.86 ± 0.21 | 4.47 ± 0.35 | 3.21 ± 0.75 |
| Example 22 | 3.37 ± 0.22 | 7.02 ± 0.27 | 5.42 ± 0.53 |
| Example 23 | 5.49 ± 0.16 | 4.62 ± 0.20 | 3.15 ± 0.46 |
| Example 24 | 64.23 ± 3.41 | 27.75 ± 1.12 | 24.26 ± 0.92 |
| Example 25 | 61.71 ± 2.97 | 34.42 ± 1.89 | 39.69 ± 2.31 |
| Example 26 | 15.41 ± 0.88 | 40.07 ± 1.57 | 32.08 ± 2.03 |
| Example 27 | 59.33 ± 2.18 | 89.03 ± 0.59 | 51.21 ± 0.87 |
| Example 28 | 47.19 ± 2.71 | 53.75 ± 3.58 | 39.54 ± 1.03 |
| Example 29 | 53.16 ± 3.47 | 19.72 ± 0.89 | 29.01 ± 2.26 |
| Example 30 | 2.95 ± 0.31 | 20.96 ± 0.49 | 15.02 ± 0.85 |
| Example 31 | 21.75 ± 0.99 | 41.60 ± 1.37 | 33.03 ± 1.13 |
| Example 32 | 28.21 ± 1.19 | 19.03 ± 0.36 | 26.58 ± 2.19 |
| Example 33 | 4.21 ± 0.15 | 5.76 ± 0.33 | 3.69 ± 0.63 |
| Example 34 | 28.73 ± 0.54 | 19.07 ± 0.78 | 11.25 ± 0.72 |

Pharmacological tests have proved that the scutellarin amide derivatives of the present disclosure have good anti-proliferative activity on tumor cell lines, especially on liver cancer cell lines, and all have higher activity than the lead compound scutellarin, and can be used for the further preparation of anti-tumor drugs.

What is claimed is:

1. A scutellarin amide derivative of formula I and a pharmaceutically acceptable salt thereof: 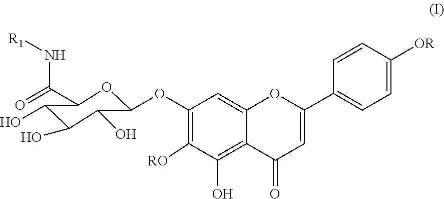
wherein R is a substituted or unsubstituted C₁-C₁₂ alkyl group, a substituted or unsubstituted benzyl group on the benzene ring, and the substituent is a C₁-C₄ alkyl group, a C₁-C₄ alkoxy group; R₁ is a substituted or unsubstituted C₁-C₁₂ alkyl group, a substituted or unsubstituted phenyl group, and the substituent is halogen, a C₁-C₄ alkyl group or a C₁-C₄ alkoxy group.

2. The compound according to claim 1, wherein R is a substituted or unsubstituted C₁-C₆ alkyl group, a substituted or unsubstituted benzyl group on the benzene ring, and the substituent is a C₁-C₄ alkyl group, a C₁-C₄ alkoxy group; R₁ is a substituted or unsubstituted C₁-C₆ alkyl group, a substituted or unsubstituted phenyl group, and the substituent is halogen, a C₁-C₄ alkyl group or a C₁-C₄ alkoxy group.

3. The compound according to claim 1, wherein R is methyl, ethyl, n-propyl, isopropyl, n-butyl, isobutyl or benzyl; R₁ is ethyl, n-propyl, isopropyl, n-butyl, n-pentyl, n-hexyl, cyclohexyl, phenyl, 4-chlorophenyl, 3-chlorophenyl, 2-chlorophenyl, 4-methylphenyl, 3-methylphenyl, 2-methylphenyl, 4-hydroxyphenyl, 3-hydroxyphenyl, 2-hydroxyphenyl, 4-methoxyphenyl, 3-methoxyphenyl, 2-methoxyphenyl.

4. The compound according to claim 1, wherein the scutellarin amide derivative of formula I and a pharmaceutically acceptable salt thereof are selected from:

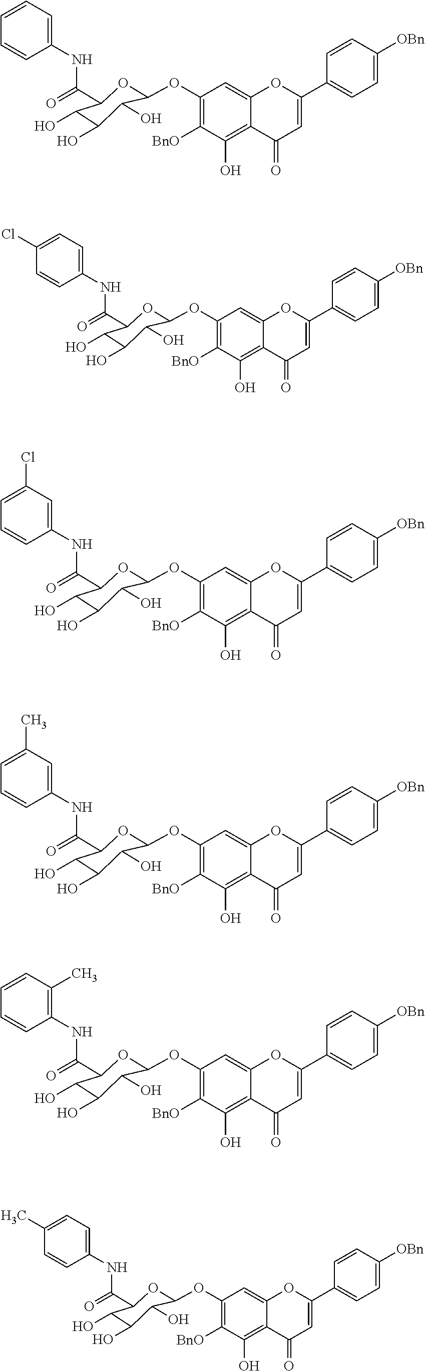

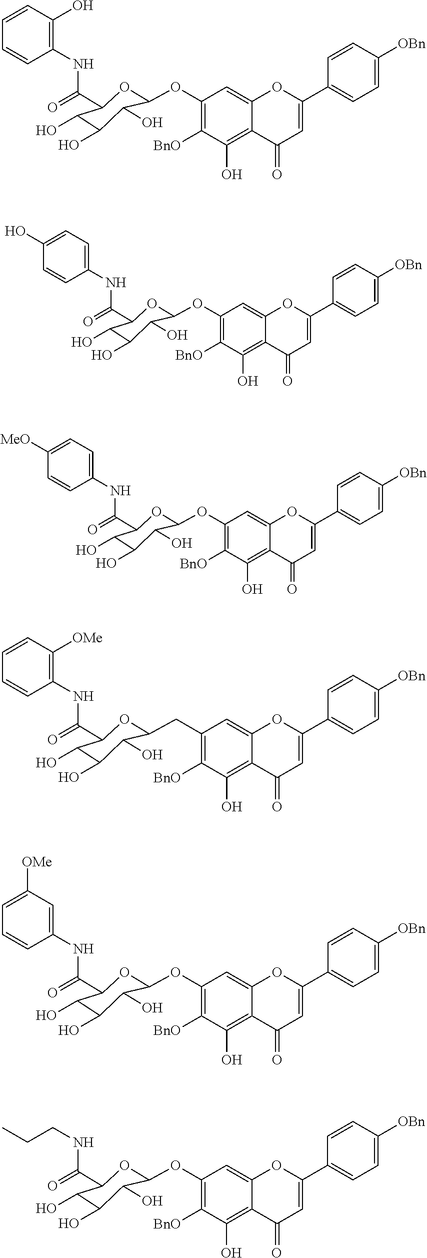

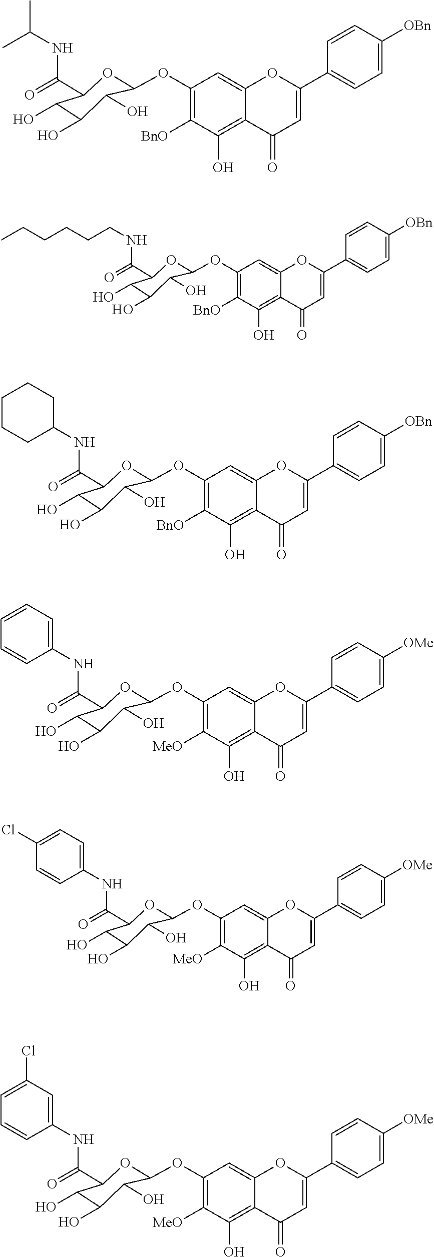 [SMILES: CC(C)NC(=O)C1OC(Oc2cc3oc(-c4ccc(OCc5ccccc5)cc4)cc(=O)c3c(O)c2OCc2ccccc2)C(O)C(O)C1O ; CCCCCCNC(=O)C1OC(Oc2cc3oc(-c4ccc(OCc5ccccc5)cc4)cc(=O)c3c(O)c2OCc2ccccc2)C(O)C(O)C1O ; O=C(NC1CCCCC1)C1OC(Oc2cc3oc(-c4ccc(OCc5ccccc5)cc4)cc(=O)c3c(O)c2OCc2ccccc2)C(O)C(O)C1O ; COc1ccc(-c2cc(=O)c3c(O)c(OC)c(OC4OC(C(=O)Nc5ccccc5)C(O)C(O)C4O)cc3o2)cc1 ; COc1ccc(-c2cc(=O)c3c(O)c(OC)c(OC4OC(C(=O)Nc5ccc(Cl)cc5)C(O)C(O)C4O)cc3o2)cc1 ; COc1ccc(-c2cc(=O)c3c(O)c(OC)c(OC4OC(C(=O)Nc5cccc(Cl)c5)C(O)C(O)C4O)cc3o2)cc1]

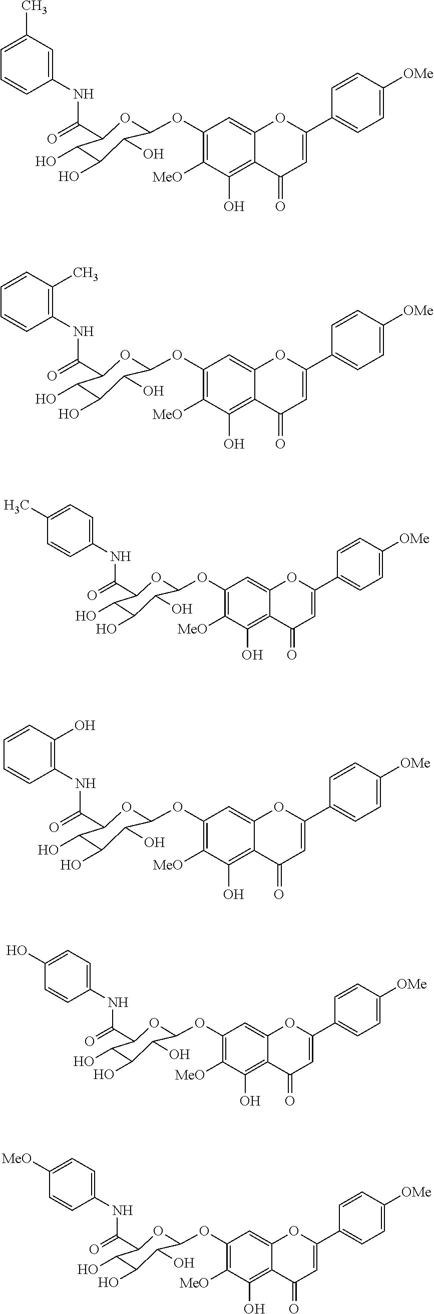 [SMILES: COc1ccc(-c2cc(=O)c3c(O)c(OC)c(OC4OC(C(=O)Nc5cccc(C)c5)C(O)C(O)C4O)cc3o2)cc1 ; COc1ccc(-c2cc(=O)c3c(O)c(OC)c(OC4OC(C(=O)Nc5ccccc5C)C(O)C(O)C4O)cc3o2)cc1 ; COc1ccc(-c2cc(=O)c3c(O)c(OC)c(OC4OC(C(=O)Nc5ccc(C)cc5)C(O)C(O)C4O)cc3o2)cc1 ; COc1ccc(-c2cc(=O)c3c(O)c(OC)c(OC4OC(C(=O)Nc5ccccc5O)C(O)C(O)C4O)cc3o2)cc1 ; COc1ccc(-c2cc(=O)c3c(O)c(OC)c(OC4OC(C(=O)Nc5ccc(O)cc5)C(O)C(O)C4O)cc3o2)cc1 ; COc1ccc(NC(=O)C2OC(Oc3cc4oc(-c5ccc(OC)cc5)cc(=O)c4c(O)c3OC)C(O)C(O)C2O)cc1]

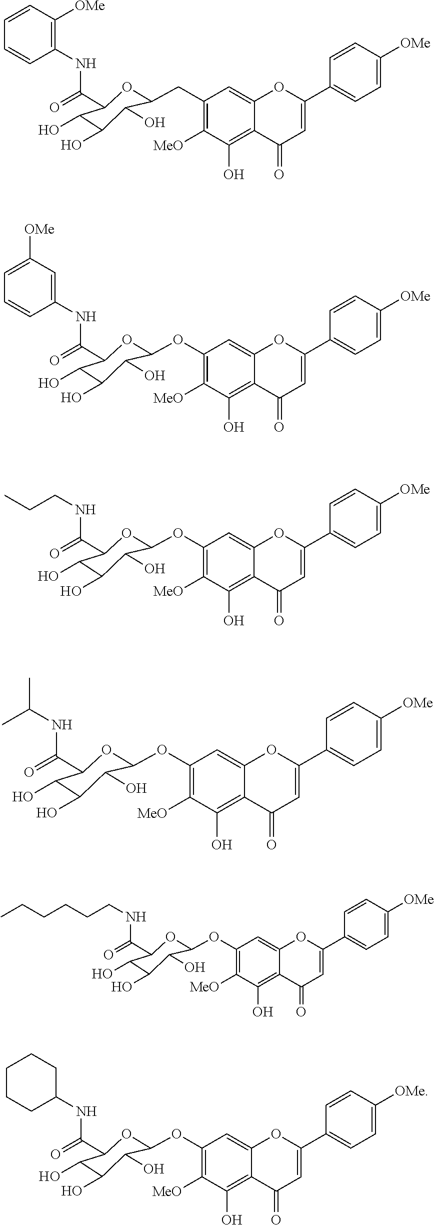 [SMILES: COc1ccc(-c2cc(=O)c3c(O)c(OC)c(CC4OC(C(=O)Nc5ccccc5OC)C(O)C(O)C4O)cc3o2)cc1 ; COc1ccc(-c2cc(=O)c3c(O)c(OC)c(OC4OC(C(=O)Nc5cccc(OC)c5)C(O)C(O)C4O)cc3o2)cc1 ; CCCNC(=O)C1OC(Oc2cc3oc(-c4ccc(OC)cc4)cc(=O)c3c(O)c2OC)C(O)C(O)C1O ; COc1ccc(-c2cc(=O)c3c(O)c(OC)c(OC4OC(C(=O)NC(C)C)C(O)C(O)C4O)cc3o2)cc1 ; CCCCCCNC(=O)C1OC(Oc2cc3oc(-c4ccc(OC)cc4)cc(=O)c3c(O)c2OC)C(O)C(O)C1O ; COc1ccc(-c2cc(=O)c3c(O)c(OC)c(OC4OC(C(=O)NC5CCCCC5)C(O)C(O)C4O)cc3o2)cc1]

5. A pharmaceutical composition comprising the compound according to claim 1 and a pharmaceutically acceptable salt thereof.

6. The pharmaceutical composition according to claim 5, wherein R is a substituted or unsubstituted C₁-C₆ alkyl group, a substituted or unsubstituted benzyl group on the benzene ring, and the substituent is a C₁-C₄ alkyl group, a C₁-C₄ alkoxy group; R₁ is a substituted or unsubstituted C₁-C₆ alkyl group, a substituted or unsubstituted phenyl group, and the substituent is halogen, a C₁-C₄ alkyl group or a C₁-C₄ alkoxy group.

7. The pharmaceutical composition according to claim 5, wherein R is methyl, ethyl, n-propyl, isopropyl, n-butyl, isobutyl or benzyl; R₁ is ethyl, n-propyl, isopropyl, n-butyl, n-pentyl, n-hexyl, cyclohexyl, phenyl, 4-chlorophenyl, 3-chlorophenyl, 2-chlorophenyl, 4-methylphenyl, 3-methylphenyl, 2-methylphenyl, 4-hydroxyphenyl, 3-hydroxyphenyl, 2-hydroxyphenyl, 4-methoxyphenyl, 3-methoxyphenyl, 2-methoxyphenyl.

8. The pharmaceutical composition according to claim 5, wherein the scutellarin amide derivative of formula I and a pharmaceutically acceptable salt thereof are selected from:

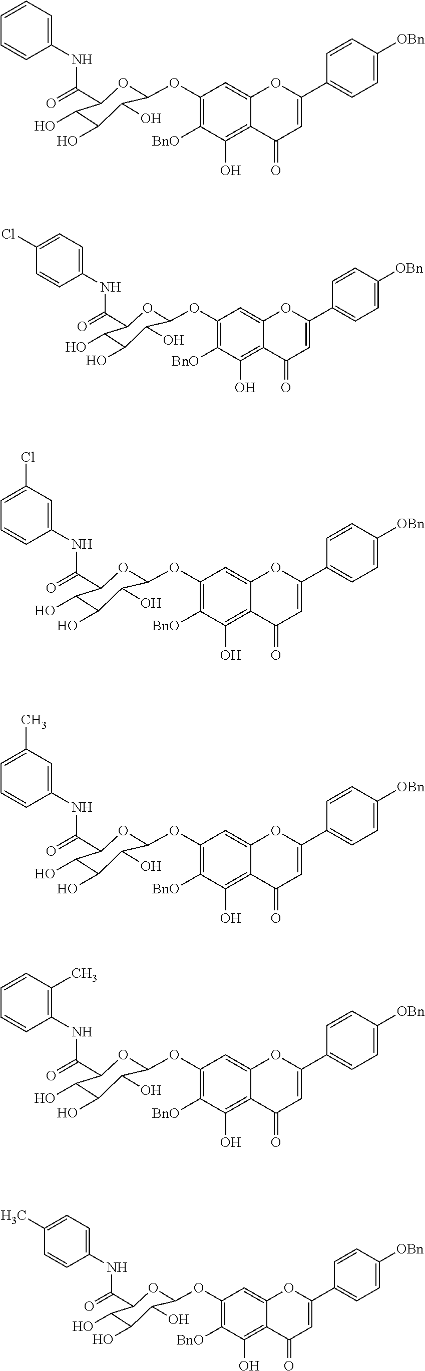

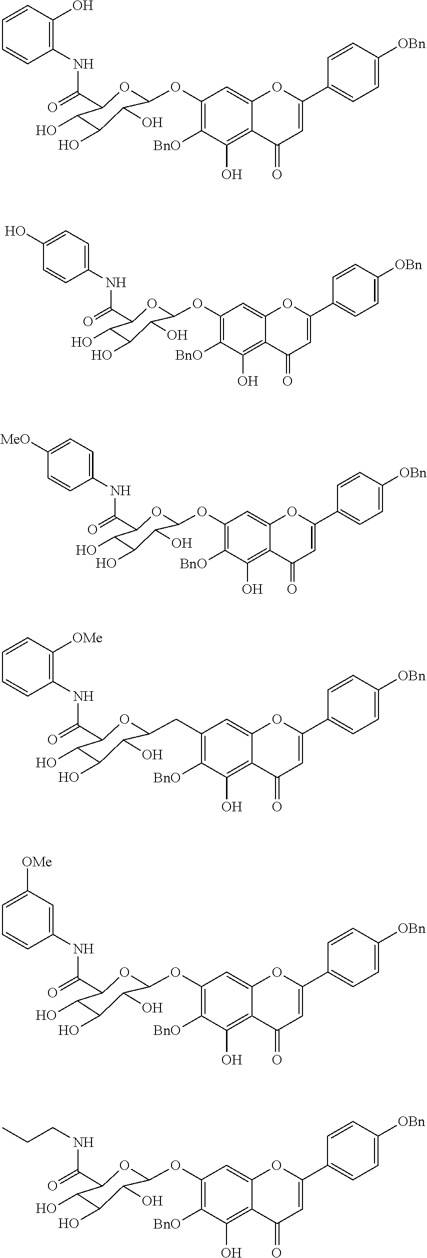

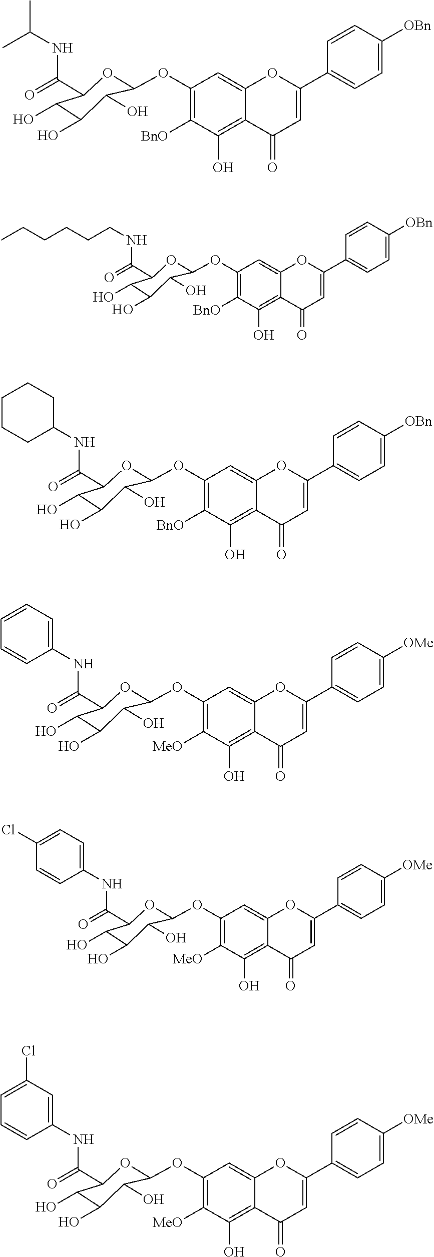 [SMILES: CC(C)NC(=O)C1OC(Oc2cc3oc(-c4ccc(OCc5ccccc5)cc4)cc(=O)c3c(O)c2OCc2ccccc2)C(O)C(O)C1O ; CCCCCCNC(=O)C1OC(Oc2cc3oc(-c4ccc(OCc5ccccc5)cc4)cc(=O)c3c(O)c2OCc2ccccc2)C(O)C(O)C1O ; O=C(NC1CCCCC1)C1OC(Oc2cc3oc(-c4ccc(OCc5ccccc5)cc4)cc(=O)c3c(O)c2OCc2ccccc2)C(O)C(O)C1O ; COc1ccc(-c2cc(=O)c3c(O)c(OC)c(OC4OC(C(=O)Nc5ccccc5)C(O)C(O)C4O)cc3o2)cc1 ; COc1ccc(-c2cc(=O)c3c(O)c(OC)c(OC4OC(C(=O)Nc5ccc(Cl)cc5)C(O)C(O)C4O)cc3o2)cc1 ; COc1ccc(-c2cc(=O)c3c(O)c(OC)c(OC4OC(C(=O)Nc5cccc(Cl)c5)C(O)C(O)C4O)cc3o2)cc1]

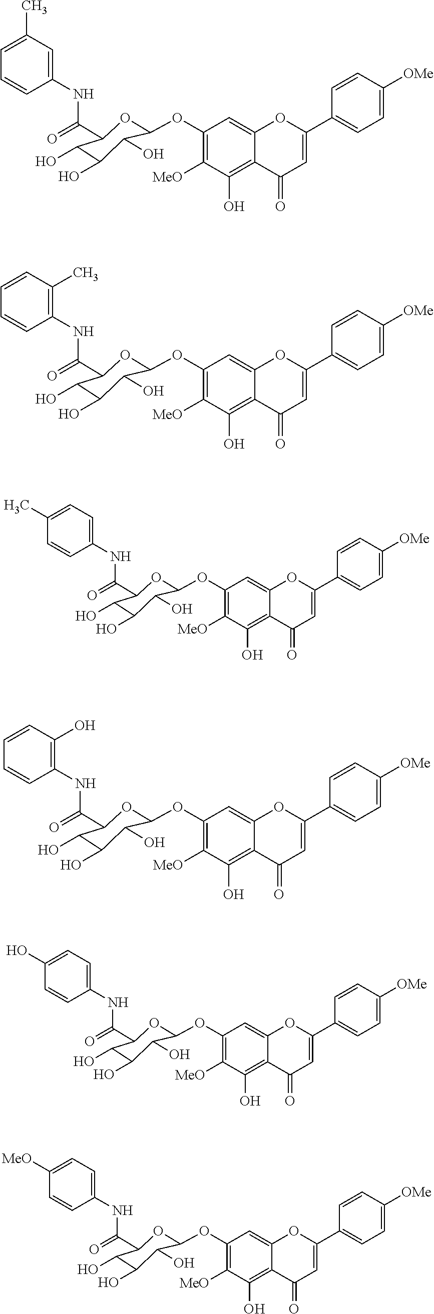 [SMILES: COc1ccc(-c2cc(=O)c3c(O)c(OC)c(OC4OC(C(=O)Nc5cccc(C)c5)C(O)C(O)C4O)cc3o2)cc1 ; COc1ccc(-c2cc(=O)c3c(O)c(OC)c(OC4OC(C(=O)Nc5ccccc5C)C(O)C(O)C4O)cc3o2)cc1 ; COc1ccc(-c2cc(=O)c3c(O)c(OC)c(OC4OC(C(=O)Nc5ccc(C)cc5)C(O)C(O)C4O)cc3o2)cc1 ; COc1ccc(-c2cc(=O)c3c(O)c(OC)c(OC4OC(C(=O)Nc5ccccc5O)C(O)C(O)C4O)cc3o2)cc1 ; COc1ccc(-c2cc(=O)c3c(O)c(OC)c(OC4OC(C(=O)Nc5ccc(O)cc5)C(O)C(O)C4O)cc3o2)cc1 ; COc1ccc(NC(=O)C2OC(Oc3cc4oc(-c5ccc(OC)cc5)cc(=O)c4c(O)c3OC)C(O)C(O)C2O)cc1]

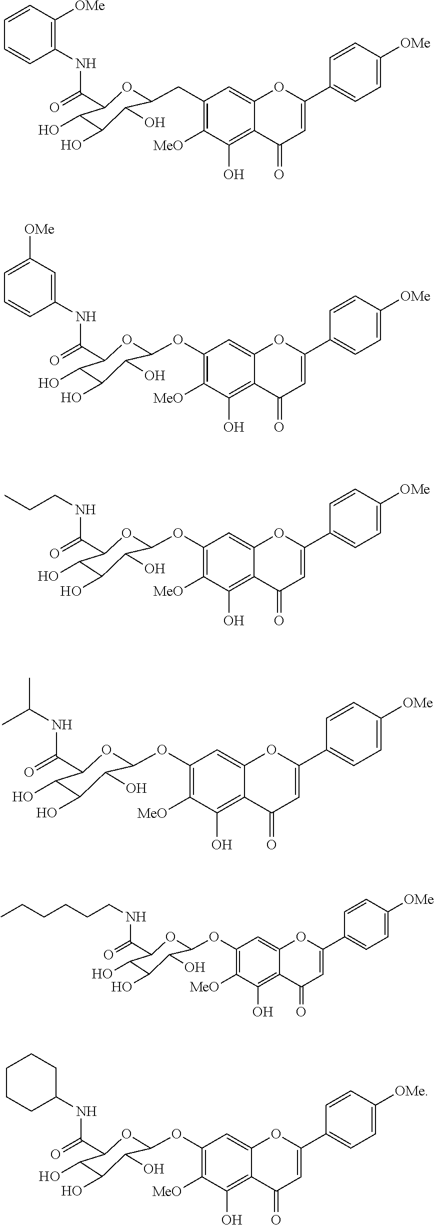 [SMILES: COc1ccc(-c2cc(=O)c3c(O)c(OC)c(CC4OC(C(=O)Nc5ccccc5OC)C(O)C(O)C4O)cc3o2)cc1 ; COc1ccc(-c2cc(=O)c3c(O)c(OC)c(OC4OC(C(=O)Nc5cccc(OC)c5)C(O)C(O)C4O)cc3o2)cc1 ; CCCNC(=O)C1OC(Oc2cc3oc(-c4ccc(OC)cc4)cc(=O)c3c(O)c2OC)C(O)C(O)C1O ; COc1ccc(-c2cc(=O)c3c(O)c(OC)c(OC4OC(C(=O)NC(C)C)C(O)C(O)C4O)cc3o2)cc1 ; CCCCCCNC(=O)C1OC(Oc2cc3oc(-c4ccc(OC)cc4)cc(=O)c3c(O)c2OC)C(O)C(O)C1O ; COc1ccc(-c2cc(=O)c3c(O)c(OC)c(OC4OC(C(=O)NC5CCCCC5)C(O)C(O)C4O)cc3o2)cc1]

9. A method of preparing a scutellarin amide derivative of the general formula I and a pharmaceutically acceptable salt thereof according to claim 1, wherein the method comprises the following steps:
Step 1: Scutellarin is reacted with a corresponding halogenated hydrocarbon in the presence of K₂CO₃/DMF for 24-48 hours, and then subjected to a silica gel column chromatography with 10:1 to 50:1 dichloromethane-methanol as the eluent to obtain intermediate 2 and intermediate 3; subsequently, the intermediates 2 and 3 are hydrolyzed in the presence of KOH/MeOH to obtain target compound 4 and target compound 5;
Step 2: The glycosylcarboxyl position of the target compounds 4, 5 is reacted with an aniline substituted by different substituents and substituted at different positions and an aliphatic amine with different side chains at room temperature for 12-24 hours in the presence of HOBt, EDCI catalysts, and then subjected to a silica gel column chromatography with 10:1 to 100:1 dichloromethane-methanol as the eluent to obtain target compounds 6a-o and 7a-o; [chemical structure]
